(19) United States
(12) Patent Application Publication
(10) Pub. No.: US 2022/0336076 A1
(43) Pub. Date: Oct. 20, 2022

(54) MEASURING PARAMETERS ASSOCIATED WITH DRUG ADMINISTRATION AND DRUG ADMINISTRATION DEVICES INCORPORATING SAME

(71) Applicant: JANSSEN PHARMACEUTICALS, INC., Titusville, NJ (US)

(72) Inventors: Francesco N. ALBERTINI, Pleasanton, CA (US); Gregory J. BAKOS, Mason, OH (US); Anthony R. DIUBALDI, Jackson, NJ (US); Jason L. HARRIS, Lebanon, OH (US); Emma Louise HUBERT, San Jose, CA (US); Michael HUTCHINSON, King of Prussia, PA (US); David KALIKHMAN, Hungtingdon Valley, PA (US); Monica A. KAPIL, San Jose, CA (US); Peter KRULEVITCH, Pleasanton, CA (US); Wouter Jacques Noel LEQUIEU, Lievegem (BE); Frederick E. SHELTON, IV, Cincinnati, OH (US); Jaskaran SINGH, San Diego, CA (US); George SZABO, Hatfield, PA (US); Steven M. VESOLE, San Bruno, CA (US); Jingli WANG, San Jose, CA (US)

(21) Appl. No.: 17/763,600

(22) PCT Filed: Sep. 24, 2020
(86) PCT No.: PCT/IB2020/058969

§ 371 Date: Mar. 24, 2022

Related U.S. Application Data
(60) Provisional application No. 62/905,454, filed on Sep. 25, 2019.
(60) Provisional application No. 62/905,457, filed on Sep. 25, 2019.
(60) Provisional application No. 62/905,460, filed on Sep. 25, 2019.
(60) Provisional application No. 63/020,942, filed on May 6, 2020.

(51) Int. Cl. G16H 20/17; A61B 5/00; A61M 5/142; A61M 5/168; G16H 20/13

(52) U.S. Cl. CPC G16H 20/17; A61B 5/0002; A61M 5/142; A61M 5/16804; G16H 20/13

(57) ABSTRACT

The present disclosure relates to drug administration. In an exemplary embodiment, a system can include a drug administration device configured to dispense a drug to a patient, a monitoring device configured to log a delivery event of drug delivery from the drug administration device into the patient, and a sensor configured to sense a patient parameter following delivery of the drug into the patient. In another exemplary embodiment, a drug administration device can include a drug holder configured to hold a drug, a dispensing mechanism configured to dispense the drug, and a sensor configured to sense a patient parameter, and the drug administration device can be configured to locally activate the drug at a target location in the patient. In another exemplary embodiment, methods, devices, and systems are provided to assess when operation of a drug dispensing mechanism is complete and to confirm whether drug administration was successful.

### FIELD

The embodiments described herein relate to a device for administering and/or provision of a drug. The present disclosure further relates to a system in which the device can be used, and a method of administration, and a further method associated with the system.

### BACKGROUND

Pharmaceutical products (including large and small molecule pharmaceuticals, hereinafter “drugs”) are administered to patients in a variety of different ways for the treatment of specific medical indications. Regardless of the manner of the administration, care must be taken when administering drugs to avoid adverse effects on the patient. For example, care must be taken not to administer more than a safe amount of the drug to the patient. This requires consideration of the amount of dose given and the time frame over which the dose is delivered, sometimes in relation to previous doses, or doses of other drugs. Moreover, care must be taken not to inadvertently administer an incorrect drug to the patient, or drugs that have degraded due to their age or storage conditions. All of these considerations can be conveyed in guidance associated with the specific drugs or drug combinations. However, this guidance is not always followed correctly, for example due to mistakes, such as human error. This can lead to adverse effects on the patient or result in inappropriate drug administration, for example insufficient or excessive volume of drug being administered for the specific medical indication.

Further, a drug administration device may operate, but may not fully complete operation, or may not successfully administer the drug. This lack of fully complete operation and unsuccessful administration may each risk harming the patient if the problem is not identified quickly.

In relation to how a drug is administered to the patient, there are various dosage forms that can be used. For example, these dosage forms may include parenteral, inhalational, oral, ophthalmic, nasal, topical, and suppository forms of one or more drugs.

The dosage forms can be administered directly to the patient via a drug administration device. There are a number of different types of drug administration devices commonly available for delivery of the various dosage forms including: syringes, injection devices (e.g., autoinjectors, jet injectors, and infusion pumps), nasal spray devices, and inhalers.

### SUMMARY

In one aspect, a method for confirming administration from a drug administration device is provided that in one embodiment includes operating a dispensing mechanism of the drug administration device; measuring at least one dispensing mechanism parameter; determining whether the operation of the dispensing mechanism is complete based on the at least one dispensing mechanism parameter; measuring at least one administration parameter; and when the operation of the dispensing mechanism is determined to be complete, comparing the measured at least one administration parameter with acceptable administration parameters in order to confirm whether the administration was successful.

The method can have any number of variations. For example, the method can further include modifying further operation of the drug administration device based on the at least one dispensing mechanism parameter and/or the at least one administration parameter. In at least some embodiments, the method can also include notifying a user that the further operation of the drug administration device has been modified. Notifying the user that the further operation of the drug administration device has been modified can include one or more of a visual feedback, an auditory feedback, and a tactile feedback. Modifying the further operation of the drug administration device can include preventing the further operation of the drug administration device when the successful administration was not confirmed. Modifying the further operation of the drug administration device can include modifying a dosage volume to be administered during further operation of the drug administration device, modifying a frequency with which a drug is administered by the drug administration device, modifying a maximum number of drug doses possible for delivery from the drug administration device, and/or modifying a rate with which a drug is administered by the drug administration device.

For still another example, measuring the at least one dispensing mechanism parameter or measuring the at least one administration parameter can include measuring a speed of a motor of the drug administration device and/or a duration of operation of the motor.

For yet another example, operating the dispensing mechanism of the drug administration device can include displacing a displaceable component from a first position of the displaceable component. In at least some embodiments, measuring the at least one dispensing mechanism parameter or the at least one administration parameter can include measuring the displacement of the displaceable component. Measuring the displacement of the displaceable component can include using a Hall effect sensor.

For another example, measuring the at least one dispensing mechanism parameter or the at least one administration parameter can include measuring a flow rate of a drug administered by the drug administration device. For yet another example, measuring the at least one administration parameter can include determining an amount of liquid present in a vicinity of an injection site. For another example, measuring the at least one administration parameter can include measuring a physiological parameter, of a user of the drug administration device, associated with successful administration.

For still another example, the method can include assessing an operational status of the drug administration device before and/or during operation of the dispensing mechanism. In at least some embodiments, assessing the operational status of the drug administration device can include at least one of analyzing a power source of the drug administration device to verify that the power source has sufficient charge for successful administration, and sensing an angular orientation of the drug administration device relative to a user of the drug administration device and determining whether the sensed angular orientation is a proper angular orientation. Assessing the operational status of the drug administration device can include moving the displaceable component of the drug administration device a predefined distance.

For another example, the method can include notifying a user whether the administration was successful. In at least some embodiments, notifying the user whether the administration was successful can include one or more of a visual feedback, an auditory feedbacks and a tactile feedback.

For yet another example, the acceptable administration parameters can include a predefined range of values, and the comparing can include determining whether the measured at least one administration parameter is within the predefined range of values. For another example, the acceptable administration parameters can include a predefined threshold value, and the comparing can include determining whether the measured at least one administration parameter is above the predefined threshold value. For still another example, the acceptable administration parameters can include a predefined threshold value, and the comparing can include determining whether the measured at least one administration parameter is below the predefined threshold value.

For another example, the drug can include at least one of infliximab, golimumab, ustekinumab, daratumumab, guselkumab, epoetin alfa, risperidone, esketamine, ketamine, and paliperidone palmitate.

In another embodiment, a method for confirming administration from a drug administration device includes operating a dispensing mechanism of the drug administration device; measuring at least one dispensing mechanism parameter; determining whether the operation of the dispensing mechanism is complete based on the at least one dispensing mechanism parameter; determining at least one physiological parameter of a user based on the at least one dispensing mechanism parameter; and when the operation of the dispensing mechanism is determined to be complete, comparing the at least one physiological parameter with acceptable physiological parameters in order to confirm whether the administration was successful.

The method can vary in any number of ways. For example, measuring the at least one dispensing mechanism parameter can include measuring a flow rate of a drug, and the at least one physiological parameter can be a heart rate of the user.

For another example, the method can further include modifying further operation of the drug administration device based on the at least one dispensing mechanism parameter and/or the at least one physiological parameter. In at least some embodiments, the method can also include notifying a user that the further operation of the drug administration device has been modified. Notifying the user that the further operation of the drug administration device has been modified can include one or more of a visual feedback, an auditory feedback, and a tactile feedback. Modifying the further operation of the drug administration device can include preventing the further operation of the drug administration device when the successful administration was not confirmed. Modifying the further operation of the drug administration device can include modifying a dosage volume to be administered during further operation of the drug administration device, modifying a frequency with which a drug is administered by the drug administration device, modifying a maximum number of drug doses possible for delivery from the drug administration device, and/or modifying a rate with which a drug is administered by the drug administration device.

For yet another example, operating the dispensing mechanism of the drug administration device can include displacing a displaceable component from a first position of the displaceable component.

For still another example, the method can further include assessing an operational status of the drug administration device before operating the dispensing mechanism. In at least some embodiments, assessing the operational status of the drug administration device can include at least one of analyzing a power source of the drug administration device to verify that the power source has sufficient charge for successful administration, and sensing an angular orientation of the drug administration device relative to a user of the drug administration device and determining whether the sensed angular orientation is a proper angular orientation. Assessing the operational status of the drug administration device can include moving the displaceable component of the drug administration device a predefined distance.

For another example, the method can further include notifying a user whether the administration was successful. In at least some embodiments, notifying the user whether the administration was successful can include one or more of a visual feedback, an auditory feedback, and a tactile feedback.

For yet another example, the drug can include at least one of infliximab, golimumab, ustekinumab, daratumumab, guselkumab, epoetin alfa, risperidone, esketamine, ketamine, and paliperidone palmitate.

In another aspect, a drug administration system is provided that in one embodiment includes a drug administration device that includes a dispensing mechanism configured to dispense a drug; and at least one sensor configured to measure at least one dispensing mechanism parameter and output dispensing mechanism data relating to the at least one dispensing mechanism parameter. The system is configured to determine whether operation of the dispensing mechanism is complete based on the dispensing mechanism data. The system also includes at least one sensor configured to measure at least one administration parameter and output administration data relating to the at least one administration parameter. The system is configured such that when the operation of the dispensing mechanism is determined to be complete, the system compares the administration data with acceptable administration data in order to confirm whether the administration was successful.

The drug administration system can vary in any number of ways. For example, the system can further include a first processor, and the first processor can be configured to receive the dispensing mechanism data and to determine whether the operation of the dispensing mechanism is complete based on the dispensing mechanism data. In at least some embodiments, the system can also include a second processor, and the second processor can be configured to receive the administration data and confirm whether the administration was successful when the operation of the dispensing mechanism is determined to be complete by the first processor. The second processor can be configured to modify further operation of the drug administration device based on the dispensing mechanism data and/or the administration data. The device can also include an indicator configured to inform a user of the drug administration device that the further operation of the drug administration device has been modified. The indicator can be configured to provide one or more of visual feedback, auditory feedback, and tactile feedback. The second processor can be configured to modify the further operation of the drug administration device, and the second processor can be configured to prevent the further operation of the drug administration device when the successful administration was not confirmed. The second processor can be configured to modify the further operation of the drug administration device, and the second processor can be configured to modify a dosage volume to be administered in any further operation of the drug administration device, to modify a frequency with which the drug is administered by the drug administration device, to modify a maximum number of drug doses possible for delivery from the drug administration device, and/or to modify a rate with which the drug is administered by the drug administration device.

For another example, the drug administration device can further include a motor, and one of the at least one dispensing sensor and the at least one administration sensor can be configured to measure the speed of the motor and/or the duration of operation of the motor. For yet another example, the at least one sensor configured to measure at least one dispensing mechanism parameter or the at least one sensor configured to measure at least one administration parameter can include a Hall effect sensor. For still another example, the at least one sensor configured to measure at least one dispensing mechanism parameter or the at least one sensor configured to measure at least one administration parameter can include a volumetric flow meter. For another example, the at least one sensor configured to measure at least one administration parameter can include a liquid detection sensor configured to measure the amount of liquid present in the vicinity of an injection site. For yet another example, the at least one sensor configured to measure at least one administration parameter can be configured to measure a physiological parameter of a user of the drug administration device, associated with successful administration.

For another example, the processor can be configured to assess an operational status of the drug administration device before and/or while the drug dispensing mechanism dispenses the drug. In at least some embodiments, the drug administration device can further include a power source, and the processor can be configured to assess the operational status of the drug administration device by verifying that the power source has sufficient charge for dispensing of the drug. The dispensing mechanism can further include a displaceable component, and the processor can be configured to assess the operational status of the drug administration device by moving the displaceable component a predefined distance.

For still another example, the system can include an indicator configured to inform a user of the drug administration device whether the administration was successful. In at least some embodiments, the indicator can be configured to provide visual feedback, auditory feedback, or tactile feedback.

For another example, the drug can include at least one of infliximab, golimumab, ustekinumab, daratumumab, guselkumab, epoetin alfa, risperidone, esketamine, ketamine, and paliperidone palmitate.

In another embodiment, a drug administration system is provided that includes a drug administration device that includes a drug holder configured to hold a drug; and a dispensing mechanism configured to dispense the drug to a patient. The system also includes a first sensor configured to sense a patient parameter. The drug administration system is configured to locally activate the drug at a target location in the patient after the drug has been dispensed by the dispensing mechanism and administered to the patient, and the local activation is responsive to the patient parameter and an external stimulus.

The system can vary in any number of ways. For example, the system can further include a second sensor configured to sense the external stimulus. In at least some embodiments, the first sensor and/or the second sensor can be integral with the drug administration device.

For another example, the drug administration device can be configured to delay the local activation after the drug has been administered to the patient by an amount of time such that the local activation coincides with a predicted localization time at the target location, and the predicted localization time can be based on the sensed patient parameter and the external stimulus.

For still another example, the system can further include an energy source configured to provide energy to locally activate the drug at the target location in the patient. In at least some embodiments, an amount of energy provided by the energy source can be responsive to the patient parameter and the external stimulus. The energy source can include one or more of: a light source; an ultra-sound source; an electro-magnetic field source; and a radioactive material.

For yet another example, the drug administration device can be further configured to administer a chemical activation agent to the target location in the patient to locally activate the drug. For still another example, the patient parameter sensed by the first sensor can include one or more of: temperature; pH level; a biomarker; glutathione level; skin thickness; subcutaneous tissue thickness; blood oxygen level; blood glucose level; blood pressure; heart rate; and metabolic rate.

For another example, the external stimulus can include one or more of: a user input; geographical location; ambient temperature; pressure; and ultraviolet radiation level. In at least some embodiments, the system can further include a user interface, and the external stimulus can be a user input inputted via the user interface.

For still another example, the drug administration device can be configured to administer the drug to the patient according to a drug dosing scheme. In at least some embodiments, the drug dosing scheme can specify one or more of the following drug dosing parameters: drug delivery rate; drug delivery duration; drug delivery volume; and drug delivery frequency. The drug administration device can include an autoinjector, and the drug dosing scheme can specify one or more of the following dosing parameters: a discharge nozzle advance depth of a discharge nozzle of the autoinjector during administration of the drug to the patient, a discharge nozzle velocity of the discharge nozzle of the autoinjector during administration of the drug to the patient, and a discharge nozzle acceleration of the discharge nozzle of the autoinjector during administration of the drug to the patient. The drug dosing scheme can be based on the sensed patient parameter and the external stimulus. The sensed patient parameter can include subcutaneous tissue thickness, and the drug administration device can be configured to adjust the discharge nozzle advance depth based on the sensed subcutaneous tissue thickness.

For another example, the drug administration system can be further configured to determine, based on the sensed patient parameter and/or the external stimulus, whether a likelihood of side effects associated with the drug has increased, and, if it is determined that the likelihood of side effects has increased, adjust the drug dosing scheme to reduce the dosage of the drug to be administered and/or adjust an activation means of the drug administration system to reduce local activation of the drug. The activation means can be configured to locally activate the drug. The drug administration device can further include a device indicator, and the drug administration device can be further configured to activate the device indicator if it is determined that the likelihood of side effects has increased.

For yet another example, the system can further include a drug capture and release mechanism configured to be implanted in a body of the patient.

For another example, the drug can include at least one of infliximab, golimumab, ustekinumab, daratumumab, guselkumab, epoetin alfa, risperidone, esketamine, ketamine, and paliperidone palmitate.

For yet another example, a method of administering a drug to a patient using the drug administration system can include dispensing the drug from the drug holder to administer the drug to the patient; receiving data relating to the patient parameter from the first sensor and receiving data relating to the external stimulus; comparing the received data with a lookup table; and locally activating the drug at the target location in the patient, wherein the local activation is based on the comparison with the lookup table. In at least some embodiments, the locally activation of the drug can be delayed after the dispensing of the drug by an amount of time corresponding to a localization time determined from the lookup table, and/or the drug can include at least one of infliximab, golimumab, ustekinumab, daratumumab, guselkumab, epoetin alfa, risperidone, esketamine, ketamine, and paliperidone palmitate.

In another aspect, a drug administration device a drug administration device is provided that in one embodiment includes a dispensing mechanism configured to dispense a drug; and at least one sensor configured to measure at least one dispensing mechanism parameter and output dispensing mechanism data relating to the at least one dispensing mechanism parameter. The device is configured to determine whether operation of the dispensing mechanism is complete based on the dispensing mechanism data. The device also includes at least one sensor configured to measure at least one administration parameter and output administration data relating to the at least one administration parameter. The device is configured such that when the operation of the dispensing mechanism is determined to be complete, the device compares the administration data with acceptable administration data in order to confirm whether the administration was successful.

The drug administration device can have any number of variations. For example, the device can further include a first processor, and the first processor can be configured to receive the dispensing mechanism data and to determine whether the operation of the dispensing mechanism is complete based on the dispensing mechanism data. In at least some embodiments, the device can also include a second processor, and the second processor can be configured to receive the administration data and confirm whether the administration was successful when the operation of the dispensing mechanism is determined to be complete by the first processor. The second processor can be configured to modify further operation of the drug administration device based on the dispensing mechanism data and/or the administration data. The device can also include an indicator configured to inform a user of the drug administration device that the further operation of the drug administration device has been modified. The indicator can be configured to provide one or more of visual feedback, auditory feedback, and tactile feedback. The second processor can be configured to modify the further operation of the drug administration device, and the second processor can be configured to prevent the further operation of the drug administration device when the successful administration was not confirmed. The second processor can be configured to modify the further operation of the drug administration device, and the second processor can be configured to modify a dosage volume to be administered in any further operation of the drug administration device, to modify a frequency with which the drug is administered by the drug administration device, and/or to modify a rate with which the drug is administered by the drug administration device.

For another example, the drug administration device can further include a motor, and one of the at least one dispensing sensor and the at least one administration sensor can be configured to measure the speed of the motor and/or the duration of operation of the motor. For yet another example, the at least one sensor configured to measure at least one dispensing mechanism parameter or the at least one sensor configured to measure at least one administration parameter can include a Hall effect sensor. For still another example, the at least one sensor configured to measure at least one dispensing mechanism parameter or the at least one sensor configured to measure at least one administration parameter can include a volumetric flow meter. For another example, the at least one sensor configured to measure at least one administration parameter can include a liquid detection sensor configured to measure the amount of liquid present in the vicinity of an injection site. For yet another example, the at least one sensor configured to measure at least one administration parameter can be configured to measure a physiological parameter of a user of the drug administration device, associated with successful administration.

For another example, the processor can be configured to assess an operational status of the drug administration device before and/or while the drug dispensing mechanism dispenses the drug. In at least some embodiments, the drug administration device can further include a power source, and the processor can be configured to assess the operational status of the drug administration device by verifying that the power source has sufficient charge for dispensing of the drug. The dispensing mechanism can further include a displaceable component, and the processor can be configured to assess the operational status of the drug administration device by moving the displaceable component a predefined distance.

For still another example, the device can include an indicator configured to inform a user of the drug administration device whether the administration was successful. In at least some embodiments, the indicator can be configured to provide visual feedback, auditory feedback, or tactile feedback.

For another example, the drug can include at least one of infliximab, golimumab, ustekinumab, daratumumab, guselkumab, epoetin alfa, risperidone, esketamine, ketamine, and paliperidone palmitate.

In another embodiment, a drug administration device includes a dispensing mechanism configured to dispense a drug; and at least one sensor configured to measure at least one dispensing mechanism parameter and output dispensing mechanism data relating to the at least one dispensing mechanism parameter. The device is configured to determine whether the operation of the dispensing mechanism is complete based on the dispensing mechanism data. The device also includes a processor configured to determine at least one physiological parameter of a user of the drug administration device based on the dispensing mechanism data and configured to, when the operation of the dispensing mechanism is determined to be complete, compare the at least one physiological parameter with acceptable physiological parameters in order to confirm whether the administration was successful.

The drug administration device can vary in any number of ways. For example, the at least one sensor can be configured to measure a flow rate of the drug, and the at least one physiological parameter can be a heart rate of the user.

For another example, the device can further include a second processor, and the second processor can be configured to modify further operation of the drug administration device based on the dispensing mechanism data and/or the at least one physiological parameter. In at least some embodiments, the device can also include an indicator configured to inform a user of the drug administration device that the further operation of the drug administration device has been modified. The indicator can be configured to provide one or more of visual feedback, auditory feedback, and tactile feedback. The second processor being configured to modify the further operation of the drug administration device can include the second processor being configured to prevent the further operation of the drug administration device when the successful administration was not confirmed. The second processor being configured to modify the further operation of the drug administration device can include the second processor being configured to modify a dosage volume to be administered in any further operation of the drug administration device, to modify a frequency with which the drug is administered by the drug administration device, and/or the second processor being configured to modify a rate at which the drug is administered by the drug administration device.

For still another example, the processor can be configured to assess the operational status of the drug administration device before the drug dispensing mechanism dispenses the drug. In at least some embodiments, the drug administration device can further include a power source, and the processor can be configured to assess an operational status of the drug administration device by verifying that the power source has sufficient charge for dispensing of the drug. The dispensing mechanism can further include a displaceable component, and the processor can be configured to assess the operational status of the drug administration device by moving the displaceable component a predefined distance.

For another example, the device can further include an indicator configured to inform a user of the drug administration device whether the administration was successful. In at least some embodiments, the indicator can be configured to provide visual feedback, auditory feedback, or tactile feedback.

For yet another example, the drug can include at least one of infliximab, golimumab, ustekinumab, daratumumab, guselkumab, epoetin alfa, risperidone, esketamine, ketamine, and paliperidone palmitate.

In another aspect, a drug administration and monitoring system is provided that in one embodiment includes a drug administration device configured to dispense a drug to a patient; a monitoring device configured to log at least one delivery event of drug delivery from the drug administration device into the patient; and a sensor configured to sense at least one patient parameter following the delivery of the drug into the patient.

The administration and monitoring system can have any number of variations. For example, the drug administration device, the monitoring device, and the sensor can all be integrated with each other into a single device.

For another example, the drug administration device and the monitoring device can both be integrated with each other into a single device, and the sensor can be a standalone device. In at least some embodiments, the patient sensor can be configured for in vivo monitoring of the patient in real time.

For yet another example, the drug administration device, the monitoring device, and the sensor can each be standalone discrete devices. In at least some embodiments, the patient sensor can be configured for in vivo monitoring of the patient in real time.

For still another example, the drug administration device, the monitoring device, and the sensor can each be configured to be able to be in data communication with each other. For another example, the monitoring device can be configured to receive data pertaining to drug delivery events from the drug administration device, and to receive the at least one patient parameter from the sensor.

For yet another example, the monitoring device can be configured to determine a drug response associated with the at least one drug delivery event on the patient based on the at least one patient parameter which is sensed, and to determine and store data pertaining to a patient outcome associated with the determined drug response and the at least one drug delivery event. In at least some embodiments, the patient outcome can be one or more of a time period after the at least one drug delivery event at which the drug response is sensed on the patient, an intensity of the determined drug response at a given time or over a given time period after drug administration to the patient, and a time duration for which the determined drug response in relation to the at least one drug delivery event.

For another example, the monitoring device can be further configured to generate a notification to the patient or a remote patient monitoring device based on the patient outcome. For yet another example, the at least one patient parameter sensed by the sensor can include one or more of: temperature; pH level; a biomarker; glutathione level; skin thickness; subcutaneous tissue thickness; blood oxygen level; blood glucose level; blood pressure; heart rate; and metabolic rate.

For still another example, the monitoring device can be further configured to check conformity of the at least one drug delivery event with a prescribed drug dosing scheme. In at least some embodiments, the monitoring device can be further configured to generate a notification to the patient or a remote patient monitoring device if the at least one drug delivery event does not conform to the prescribed drug dosing scheme. The drug dosing scheme can specify one or more of the following drug dosing parameters: drug delivery rate; drug delivery duration; drug delivery volume; and drug delivery frequency.

For another example, the system can include an environmental sensor configured to detect an external stimulus. In at least some embodiments, the environmental sensor can be configured to detect one or more of: a user input to the drug administration device; geographical location; ambient temperature; pressure; and ultraviolet radiation level. The system can also include a user interface, and the external stimulus can be a user input inputted via the user interface. The monitoring device can be further configured to determine, based on the sensed at least one patient parameter and/or the external stimulus, whether a likelihood of side effects associated with the drug has increased, and, if it is determined that the likelihood of side effects has increased, generate a notification to the patient or a remote patient monitoring device if the at least one drug delivery event does not conform to the prescribed drug dosing scheme. The monitoring device can include a device indicator, and the drug administration device can be further configured to activate the device indicator if it is determined that the likelihood of side effects has increased.

For yet another example, the monitoring device can be configured to provide a plurality of notifications to a patient or a remote monitoring device pertaining to the at least one drug delivery event and/or the at least one patient parameter, and the plurality of notifications can be provided in order according to a predefined priority order based on the at least one drug delivery event and/or the at least one patient parameter.

For another example, the drug can include at least one of infliximab, golimumab, ustekinumab, daratumumab, guselkumab, epoetin alfa, risperidone, esketamine, ketamine, and paliperidone palmitate.

In another aspect, a method of monitoring drug administration is provided that in one embodiment includes dispensing a drug from a drug administration device to a patient; logging at least one drug delivery event of the drug administration device into the patient; and sensing at least one patient parameter following delivery of drug into the patient and the logging of the at least one drug delivery event.

The method can have any number of variations. For another example, the drug can include at least one of infliximab, golimumab, ustekinumab, daratumumab, guselkumab, epoetin alfa, risperidone, esketamine, ketamine, and paliperidone palmitate.

### BRIEF DESCRIPTION OF DRAWINGS

The present invention is described by way of reference to the accompanying figures which are as follows:

FIG. 1 is a schematic view of a first type of drug administration device, namely an autoinjector;

FIG. 2 is a schematic view of a second type of drug administration device, namely an infusion pump;

FIG. 3 is a schematic view of a third type of drug administration device, namely an inhaler;

FIG. 4 is a schematic view of a fourth type of drug administration device, namely a nasal spray device;

FIG. 5A is a schematic view of a general drug administration device;

FIG. 5B is a schematic view of a universal drug administration device;

FIG. 6 is a schematic view of a housing for a dosage form;

FIG. 7 is a schematic view of one embodiment of a communication network system with which the drug administration devices and housing can operate;

FIG. 8 is a schematic view of one embodiment of a computer system with which the drug administration devices and housing can operate;

FIG. 9 is a schematic view of one embodiment of a drug administration device which comprises a volumetric flow meter and a Hall effect sensor;

FIG. 10 is a flow diagram of one embodiment of a method of confirming administration from a drug administration device;

FIG. 11 is a schematic view of one embodiment of a monitoring system for use with the drug administration devices and systems described herein;

FIG. 12 is a schematic view of one embodiment sensor communication for use with the drug administration devices and systems described herein;

FIG. 13 is a flow diagram of one embodiment of a notification priority matrix;

FIG. 14 is a schematic view of one embodiment of a sensor operating in conjunction with a drug administration device;

FIG. 15 is a schematic view of one embodiment of a drug holder;

FIG. 16 is a schematic view of one embodiment of a drug delivery system including the drug holder of FIG. 15;

FIG. 17 is a schematic view of one embodiment of a drug administration device configured to mix a first liquid drug and a second liquid drug;

FIG. 18 is a schematic view of one embodiment of a drug administration device configured to mix a first liquid drug and a second solid drug; and

FIG. 19 is a schematic view of one embodiment of a drug delivery system in which local activation is employed.

### DETAILED DESCRIPTION

Certain exemplary embodiments will now be described to provide an overall understanding of the principles of the structure, function, manufacture, and use of the devices, systems, and methods disclosed herein. One or more examples of these embodiments are illustrated in the accompanying drawings. A person skilled in the art will understand that the devices, systems, and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. A person skilled in the art will appreciate that a dimension may not be a precise value but nevertheless be considered to be at about that value due to any number of factors such as manufacturing tolerances and sensitivity of measurement equipment. Sizes and shapes of the systems and devices, and the components thereof, can depend at least on the size and shape of components with which the systems and devices will be used.

Examples of various types of drug administration devices, namely: an autoinjector 100, an infusion pump 200, an inhaler 300, and a nasal spray device 400, are described below with reference to the hereinbefore referenced figures.

### Autoinjector

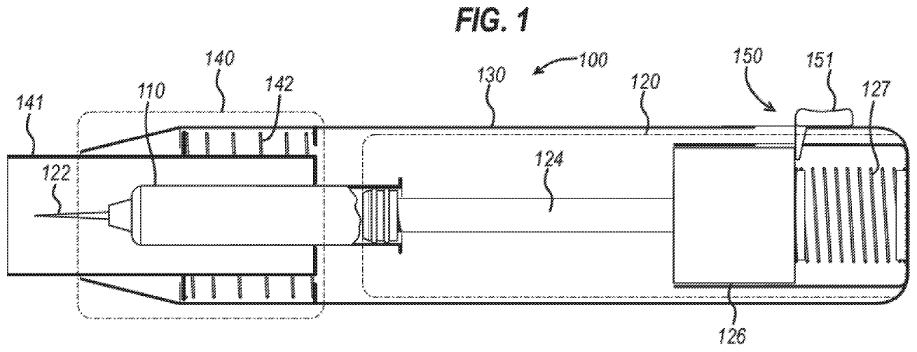

FIG. 1 is a schematic exemplary view of a first type of drug delivery device, namely an injection device, in this example an autoinjector 100, useable with embodiments described herein. The autoinjector 100 comprises a drug holder 110 which retains a drug to be dispensed and a dispensing mechanism 120 which is configured to dispense a drug from the drug holder 110 so that it can be administered to a patient. The drug holder 110 is typically in the form of a container which contains the drug, for example it may be provided in the form of a syringe or a vial, or be any other suitable container which can hold the drug. The autoinjector 100 comprises a discharge nozzle 122, for example a needle of a syringe, which is provided at a distal end of the drug holder 110. The dispensing mechanism 120 comprises a drive element 124, which itself may also comprise a piston and/or a piston rod, and drive mechanism 126. The dispensing mechanism 120 is located proximal to the end of the drug holder 110 and towards the proximal end of the autoinjector 100.

The autoinjector 100 comprises a housing 130 which contains the drug holder 110, drive element 124 and drive mechanism 126 within the body of the housing 130, as well as containing the discharge nozzle 122, which, prior to injection, would typically be contained fully within the housing, but which would extend out of the housing 130 during an injection sequence to deliver the drug. The dispensing mechanism 120 is arranged so that the drive element 124 is advanced through the drug holder 110 in order to dispense the drug through the discharge nozzle 122, thereby allowing the autoinjector to administer a drug retained in drug holder 110 to a patient. In some instances, a user may advance the drive element 124 through the drug holder 110 manually. In other instances, the drive mechanism 126 may include a stored energy source 127 which advances the drive element 124 without user assistance. The stored energy source 127 may include a resilient biasing member such as a spring, or a pressurized gas, or electronically powered motor and/or gearbox.

The autoinjector 100 includes a dispensing mechanism protection mechanism 140. The dispensing mechanism protection mechanism 140 typically has two functions. Firstly, the dispensing mechanism protection mechanism 140 can function to prevent access to the discharge nozzle 122 prior to and after injection. Secondly, the autoinjector 100 can function, such that when put into an activated state, e.g., the dispensing mechanism protection mechanism 140 is moved to an unlocked position, the dispensing mechanism 120 can be activated.

The protection mechanism 140 covers at least a part of the discharge nozzle 122 when the drug holder 110 is in its retracted position proximally within the housing 130. This is to impede contact between the discharge nozzle 122 and a user. Alternatively, or in addition, the protection mechanism 140 is itself configured to retract proximally to expose the discharge nozzle 122 so that it can be brought into contact with a patient. The protection mechanism 140 comprises a shield member 141 and return spring 142. Return spring 142 acts to extend the shield member 141 from the housing 130, thereby covering the discharge nozzle 122 when no force is applied to the distal end of the protection mechanism 140. If a user applies a force to the shield member 141 against the action of the return spring 142 to overcome the bias of the return spring 142, the shield member 141 retracts within the housing 130, thereby exposing the discharge nozzle 122. The protection mechanism 140 may alternatively, or in addition, comprise an extension mechanism (not shown) for extending the discharge nozzle 122 beyond the housing 130, and may further comprise a retracting mechanism (not shown) for retracting the discharge nozzle 122 within the housing 130. The protection mechanism 140 may alternatively, or in addition, comprise a housing cap and/or discharge nozzle boot, which can be attached to the autoinjector 100. Removal of the housing cap would typically also remove the discharge nozzle boot from the discharge nozzle 122.

The autoinjector 100 also includes a trigger 150. The trigger 150 comprises a trigger button 151 which is located on an external surface of the housing 130 so that it is accessible by a user of the autoinjector 100. When the trigger 150 is pressed by a user, it acts to release the drive mechanism 126 so that, via the drive element 124, the drug is then driven out of the drug holder 110 via the discharge nozzle 122.

The trigger 150 may also cooperate with the shield member 141 in such a way that the trigger 150 is prevented from being activated until the shield member 141 has been retracted proximally sufficiently into the housing 130 into an unlocked position, for example by pushing a distal end of the shield member 141 against the skin of a patient. When this has been done, the trigger 150 becomes unlocked, and the autoinjector 100 is activated such that the trigger 150 can be depressed and the injection and/or drug delivery sequence is then initiated. Alternatively, retraction of the shield member 141 alone in a proximal direction into the housing 130 can act to activate the drive mechanism 126 and initiate the injection and/or drug delivery sequence. In this way, the autoinjector 100 has device operation prevention mechanism which prevents dispensing of the drug by, for example, preventing accidental release of the dispensing mechanism 120 and/or accidental actuation of the trigger 150.

Whilst the foregoing description relates to one example of an autoinjector, this example is presented purely for illustration, the present invention is not limited solely to such an autoinjector. A person skilled in the art understands that various modifications to the described autoinjector may be implemented within the scope of the present disclosure.

Autoinjectors of the present disclosure can be used to administer any of a variety of drugs, such as any of epinephrine, Rebif, Enbrel, Aranesp, atropine, pralidoxime chloride, and diazepam.

### Infusion Pump

In other circumstances, patients can require precise, continuous delivery of medication or medication delivery on a regular or frequent basis at set periodic intervals. Infusion pumps can provide such controlled drug infusion, by facilitating the administering of the drug at a precise rate that keeps the drug concentration within a therapeutic margin, without requiring frequent attention by a healthcare professional or the patient.

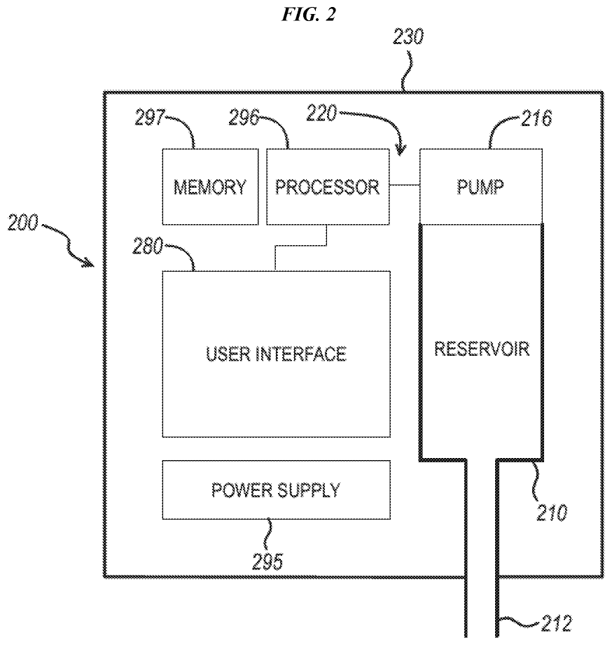

FIG. 2 is a schematic exemplary view of a second type of drug delivery device, namely an infusion pump 200, useable with the embodiments described herein. The infusion pump 200 comprises a drug holder 210 in the form of a reservoir for containing a drug to be delivered, and a dispensing mechanism 220 comprising a pump 216 adapted to dispense a drug contained in the reservoir, so that the drug can be delivered to a patient. These components of the infusion pump are located within housing 230. The dispensing mechanism 220 further comprises an infusion line 212. The drug is delivered from the reservoir upon actuation of the pump 216 via the infusion line 212, which may take the form of a cannula. The pump 216 may take the form of an elastomeric pump, a peristaltic pump, an osmotic pump, or a motor-controlled piston in a syringe. Typically, the drug is delivered intravenously, although subcutaneous, arterial and epidural infusions may also be used.

Infusion pumps of the present disclosure can be used to administer any of a variety of drugs, such as any of insulin, antropine sulfate, avibactam sodium, bendamustine hydrochloride, carboplatin, daptomycin, epinephrine, levetiracetam, oxaliplatin, paclitaxel, pantoprazole sodium, treprostinil, vasopressin, voriconazole, and zoledronic acid.

The infusion pump 200 further comprises control circuitry, for example a processor 296 in addition to a memory 297 and a user interface 280, which together provide a triggering mechanism and/or dosage selector for the pump 200. The user interface 280 may be implemented by a display screen located on the housing 230 of the infusion pump 200. The control circuitry and user interface 280 can be located within the housing 230, or external thereto and communicate via a wired or wireless interface with the pump 216 to control its operation.

Actuation of the pump 216 is controlled by the processor 296 which is in communication with the pump 216 for controlling the pump's operation. The processor 296 may be programmed by a user (e.g., patient or healthcare professional), via a user interface 280. This enables the infusion pump 200 to deliver the drug to a patient in a controlled manner. The user can enter parameters, such as infusion duration and delivery rate. The delivery rate may be set by the user to a constant infusion rate or as set intervals for periodic delivery, typically within pre-programmed limits. The programmed parameters for controlling the pump 216 are stored in and retrieved from the memory 297 which is in communication with the processor 296. The user interface 280 may take the form of a touch screen or a keypad.

A power supply 295 provides power to the pump 216, and may take the form of an energy source which is integral to the pump 216 and/or a mechanism for connecting the pump 216 to an external source of power.

The infusion pump 200 may take on a variety of different physical forms depending on its designated use. It may be a stationary, non-portable device, e.g., for use at a patient's bedside, or it may be an ambulatory infusion pump which is designed to be portable or wearable. An integral power supply 295 is particularly beneficial for ambulatory infusion pumps.

While the foregoing description relates to one example of an infusion pump, this example is provided purely for illustration. The present disclosure is not limited to such an infusion pump. A person skilled in the art understands that various modifications to the described infusion pump may be implemented within the scope of the present disclosure. For example, the processor may be pre-programmed, such that it is not necessary for the infusion pump to include a user interface.

### Inhaler

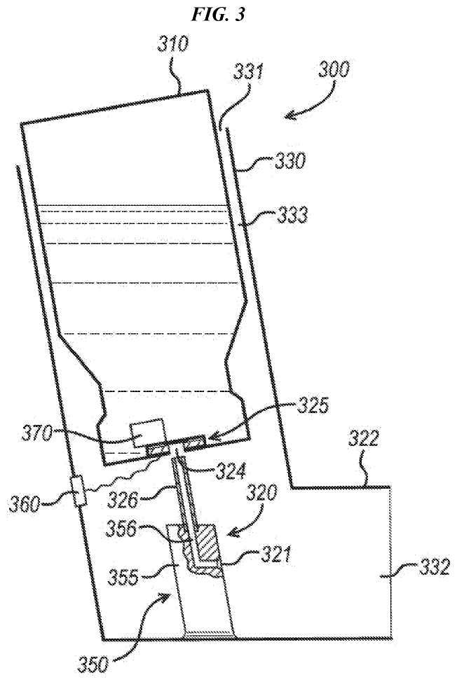

FIG. 3 is a schematic view of a third type of drug administration device, namely an inhaler 300. Inhaler 300 includes a drug holder 310 in the form of a canister. The drug holder 310 contains a drug that would typically be in solution or suspension with a suitable carrier liquid. The inhaler 300 further comprises a dispensing mechanism 320, which includes a pressurized gas for pressurizing the drug holder 310, a valve 325 and nozzle 321. The valve 325 forms an outlet of the drug holder 310. The valve 325 comprises a narrow opening 324 formed in the drug holder 310 and a movable element 326 that controls the opening 324. When the movable element 326 is in a resting position, the valve 325 is in a closed or unactuated state in which the opening 324 is closed and the drug holder 310 is sealed. When the movable element 326 is actuated from the resting position to an actuated position, the valve 325 is actuated into an open state in which the opening 324 is open. Actuation of the movable element 326 from the resting position to the actuated position comprises moving the movable element 326 into the drug holder 310. The movable element 326 is resiliently biased into the resting position. In the open state of the valve 325, the pressurized gas propels the drug in solution or suspension with the suitable liquid out of the drug holder 310 through the opening 324 at high speed. The high speed passage of the liquid through the narrow opening 324 causes the liquid to be atomized, that is, to transform from a bulk liquid into a mist of fine droplets of liquid and/or into a gas cloud. A patient may inhale the mist of fine droplets and/or the gas cloud into a respiratory passage. Hence, the inhaler 300 is capable of delivering a drug retained within the drug holder 310 into a respiratory passage of a patient.

The drug holder 310 is removably held within a housing 330 of the inhaler 300. A passage 333 formed in the housing 330 connects a first opening 331 in the housing 330 and a second opening 332 in the housing 330. The drug holder 310 is received within the passage 333. The drug holder 310 is slidably insertable through the first opening 331 of the housing 330 into the passage 333. The second opening 332 of the housing 330 forms a mouthpiece 322 configured to be placed in a patient's mouth or a nosepiece configured to be placed in a patient's nostril, or a mask configured to be placed over the patient's mouth and nose. The drug holder 310, the first opening 331 and the passage 333 are sized such that air can flow through the passage 333, around the drug holder 310, between the first opening 331 and the second opening 332. The inhaler 300 may be provided with a dispensing mechanism protection mechanism 140 in the form of a cap (not shown) which can be fitted to the mouthpiece 322.

Inhaler 300 further comprises a trigger 350 including a valve actuation feature 355 configured to actuate the valve 325 when the trigger 350 is activated. The valve actuation feature 355 is a projection of the housing 330 into the passage 333. The drug holder 310 is slidably movable within the passage 333 from a first position into a second position. In the first position, an end of the movable element 326 in the resting position abuts the valve actuation feature 355. In the second position, the drug holder 310 can be displaced towards the valve actuation feature 355 such that the valve actuation feature 355 moves the movable element 326 into the drug holder 310 to actuate the valve 325 into the open state. The user's hand provides the necessary force to move the drug holder 310 from the first position to the second position against the resiliently biased movable element 326. The valve actuation feature 355 includes an inlet 356, which is connected to the nozzle 321. The inlet 356 of the valve actuation feature 355 is sized and positioned to couple to the opening 324 of the valve 325 such that the ejected mist of droplets and/or gas cloud can enter the inlet 356 and exit from the nozzle 321 into the passage 333. The nozzle 321 assists in the atomization of the bulk liquid into the mist of droplets and/or gas cloud.

The valve 325 provides a metering mechanism 370. The metering mechanism 370 is configured to close the valve after a measured amount of liquid, and therefore, drug, has passed through the opening 324. This allows a controlled dose to be administered to the patient. Typically, the measured amount of liquid is pre-set, however, the inhaler 300 may be equipped with a dosage selector 360 that is user operable to change the defined amount of liquid.

While the foregoing description relates to one particular example of an inhaler, this example is purely illustrative. The description should not be seen as limited only to such an inhaler. A person skilled in the art understands that numerous other types of inhaler and nebulizers may be used with the present disclosure. For example, the drug may be in a powdered form, the drug may be in liquid form, or the drug may be atomized by other forms of dispensing mechanism 320 including ultrasonic vibration, compressed gas, a vibrating mesh, or a heat source.

The inhalers of the present disclosure can be used to administer any of a variety of drugs, such as any of mometasone, fluticasone, ciclesonide, budesonide, beclomethasone, vilanterol, salmeterol, formoterol, umeclidinium, glycopyrrolate, tiotropium, aclidinium, indacaterol, salmeterol, and olodaterol.

### Nasal Spray Device

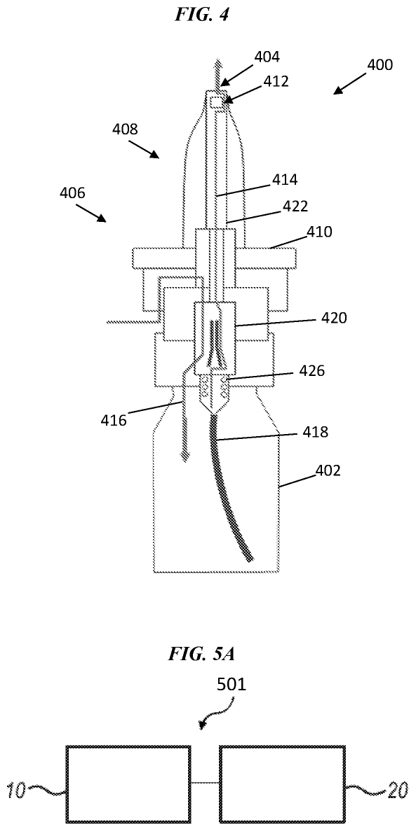

FIG. 4 is a schematic view of a fourth type of drug administration device, namely a nasal spray device 400. The nasal spray device 400 is configured to expel a drug into a nose of a patient. The nasal spray device 400 includes a drug holder 402 configured to contain a drug therein for delivery from the device 400 to a patient. The drug holder 102 can have a variety of configurations, such as a bottle reservoir, a cartridge, a vial (as in this illustrated embodiment), a blow-fill-seal (BFS) capsule, a blister pack, etc. In an exemplary embodiment, the drug holder 402 is a vial. An exemplary vial is formed of one or more materials, e.g., glass, polymer(s), etc. In some embodiments, a vial can be formed of glass. In other embodiments, a vial can be formed of one or more polymers. In yet other embodiments, different portions of a vial can be formed of different materials. An exemplary vial can include a variety of features to facilitate sealing and storing a drug therein, as described herein and illustrated in the drawings. However, a person skilled in the art will appreciate that the vials can include only some of these features and/or can include a variety of other features known in the art. The vials described herein are merely intended to represent certain exemplary embodiments.

An opening 404 of the nasal spray device 400 through which the drug exits the nasal spray device 400 is formed in in a dispensing head 406 of the nasal spray device 400 in a tip 408 of the dispensing head 406. The tip 408 is configured to be inserted into a nostril of a patient. In an exemplary embodiment, the tip 408 is configured to be inserted into a first nostril of the patient during a first stage of operation of the nasal spray device 400 and into a second nostril of the patient during a second stage of operation of the nasal spray device 400. The first and second stages of operation involve two separate actuations of the nasal spray device 400, a first actuation corresponding to a first dose of the drug being delivered and a second actuation corresponding to a second dose of the drug being delivered. In some embodiments, the nasal spray device 400 is configured to be actuated only once to deliver one nasal spray. In some embodiments, the nasal spray device 400 is configured to be actuated three or more times to deliver three or more nasal sprays, e.g., four, five, six, seven, eight, nine, ten, etc.

The dispensing head 406 includes a depth guide 410 configured to contact skin of the patient between the patient's first and second nostrils, such that a longitudinal axis of the dispensing head 406 is substantially aligned with a longitudinal axis of the nostril in which the tip 408 is inserted. A person skilled in the art will appreciate that the longitudinal axes may not be precisely aligned but nevertheless be considered to be substantially aligned due to any number of factors, such as manufacturing tolerances and sensitivity of measurement equipment.

In an exemplary embodiment, as in FIG. 4, the dispensing head 406 has a tapered shape in which the dispensing head 406 has a smaller diameter at its distal end than at its proximal end where the opening 404 is located. The opening 404 having a relatively small diameter facilitates spray of the drug out of the opening 404, as will be appreciated by a person skilled in the art. A spray chamber 412 through which the drug is configured to pass before exiting the opening 404 is located within a proximal portion of the tapered dispensing head 406, distal to the opening 404. When the drug passes through the spray chamber 412 at speed, the spray chamber 412 facilitates production of a fine mist that exits through the opening 404 with a consistent spray pattern. Arrow 414 in FIG. 4 illustrates a path of travel of the drug from the drug holder 402 and out of the opening 404.

In some embodiments, the dispensing head 406 can include two tips 408 each having an opening 404 therein such that the nasal spray device 400 is configured to simultaneously deliver doses of drug into two nostrils in response to a single actuation.

The dispensing head 406 is configured to be pushed toward the drug holder 402, e.g., depressed by a user pushing down on the depth guide 410, to actuate the nasal spray device 400. In other words, the dispensing head 406 is configured as an actuator to be actuated to drive the drug from the drug holder 402 and out of the nasal spray device 400. In an exemplary embodiment, the nasal spray device 400 is configured to be self-administered such that the user who actuates the nasal spray device 400 is the patient receiving the drug from the nasal spray device 400, although another person can actuate the nasal spray device 400 for delivery into another person.

The actuation, e.g., depressing, of the dispensing head 406 is configured to cause venting air to enter the drug holder 402, as shown by arrow 416 in FIG. 4. The air entering the drug holder 402 displaces drug in the drug holder through a tube 418 and then into a metering chamber 420, which displaces drug proximally through a cannula 422, through the spray chamber 412, and then out of the opening 404. In response to release of the dispensing head 406, e.g., a user stops pushing downward on the dispensing head 406, a bias spring 426 causes the dispensing head 406 to return to its default, resting position to position the dispensing head 406 relative to the drug holder 402 for a subsequent actuation and drug delivery.

While the foregoing description relates to one particular example of a nasal spray device, this example is purely illustrative. The description should not be seen as limited only to such a nasal spray device. A person skilled in the art understands that the nasal spray device 400 can include different features in different embodiments depending upon various requirements. For example, the nasal spray device 400 can lack the depth guide 410 and/or may include any one or more of a device indicator, a sensor, a communications interface, a processor, a memory, and a power supply.

The nasal spray devices of the present disclosure can be used to administer any of a variety of drugs, such as any of ketamine (e.g., Ketalar®), esketamine (e.g., Spravato®, Ketanest®, and Ketanest-S®), naloxone (e.g., Narcan®), and sumatriptan (e.g., Imitrex®).

### Drug Administration Device

As will be appreciated from the foregoing, various components of drug delivery devices are common to all such devices. These components form the essential components of a universal drug administration device. A drug administration device delivers a drug to a patient, where the drug is provided in a defined dosage form within the drug administration device.

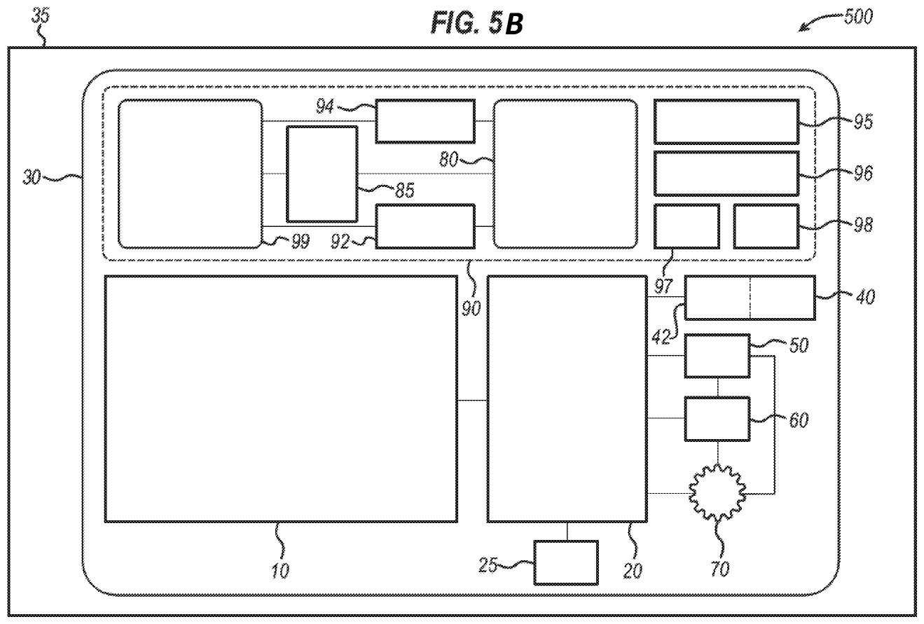

FIG. 5A is a generalized schematic view of such a universal drug administration device 501, and FIG. 5B is an exemplary embodiment of such a universal drug administration device 500. Examples of the universal drug administration device 500 include injection devices (e.g., autoinjectors, jet injectors, and infusion pumps), nasal spray devices, and inhalers.

As shown in FIG. 5A, drug administration device 501 includes in general form the features of a drug holder 10 and a dispensing mechanism 20. The drug holder 10 holds a drug in a dosage form to be administered. The dispensing mechanism 20 is configured to release the dosage form from the drug holder 10 so that the drug can be administered to a patient.

FIG. 5B shows a further universal drug administration device 500 which includes a number of additional features. A person skilled in the art understands that these additional features are optional for different embodiments, and can be utilized in a variety of different combinations such that the additional features may be present or may be omitted from a given embodiment of a particular drug administration device, depending upon requirements, such as the type of drug, dosage form of the drug, medical indication being treated with the drug, safety requirements, whether the device is powered, whether the device is portable, whether the device is used for self-administration, and many other requirements which will be appreciated by a person skilled in the art. Similar to the universal device of FIG. 4, the drug administration device 500 comprises a housing 30 which accommodates the drug holder 10 and dispensing mechanism 20.

The device 500 is provided with a triggering mechanism 50 for initiating the release of the drug from the drug holder 10 by the dispensing mechanism 20. The device 500 includes the feature of a metering/dosing mechanism 70 which measures out a set dose to be released from the drug holder 10 via the dispensing mechanism 20. In this manner, the drug administration device 500 can provide a known dose of determined size. The device 500 comprises a dosage selector 60 which enables a user to set the dose volume of drug to be measured out by the metering mechanism 50. The dose volume can be set to one specific value of a plurality of predefined discrete dose volumes, or any value of predefined dose volume within a range of dose volumes.

The device 500 can comprise a device operation prevention mechanism 40 or 25 which when in a locked state will prevent and/or stop the dispensing mechanism 20 from releasing the drug out of the drug holder 10, and when in an unlocked state will permit the dispensing mechanism 20 to release the drug dosage from out of the drug holder 10. This can prevent accidental administration of the drug, for example to prevent dosing at an incorrect time, or for preventing inadvertent actuation. The device 500 also includes a dispensing mechanism protection mechanism 42 which prevents access to at least a part of the dispensing mechanism 20, for example for safety reasons. Device operation prevention mechanism 40 and dispensing mechanism protection mechanism 42 may be the same component.

The device 500 can include a device indicator 85 which is configured to present information about the status of the drug administration device and/or the drug contained therein. The device indicator 85 may be a visual indicator, such as a display screen, or an audio indicator. The device 500 includes a user interface 80 which can be configured to present a user of the device 500 with information about the device 500 and/or to enable the user to control the device 500. The device 500 includes a device sensor 92 which is configured to sense information relating to the drug administration device and/or the drug contained therein, for example dosage form and device parameters. As an example, in embodiments which include a metering mechanism 70 and a dosage selector 60, the embodiment may further include one or more device sensors 92 configured to sense one or more of: the dose selected by a user using dosage selector 60, the dose metered by the metering mechanism 70 and the dose dispensed by the dispensing mechanism 20. Similarly, an environment sensor 94 is provided which is configured to sense information relating to the environment in which the device 500 is present, such as the temperature of the environment, the humidity of the environment, location, and time. There may be a dedicated location sensor 98 which is configured to determine the geographical location of the device 500, e.g., via satellite position determination, such as GPS. The device 500 also includes a communications interface 99 which can communicate externally data which has been acquired from the various sensors about the device and/or drug.

If required, the device 500 comprises a power supply 95 for delivering electrical power to one or more electrical components of the device 500. The power supply 95 can be a source of power which is integral to device 500 and/or a mechanism for connecting device 500 to an external source of power. The drug administration device 500 also includes a device computer system 90 including processor 96 and memory 97 powered by the power supply 95 and in communication with each other, and optionally with other electrical and control components of the device 500, such as the environment sensor 94, location sensor 98, device sensor 92, communications interface 99, and/or indicator 85. The processor 96 is configured to obtain data acquired from the environment sensor 94, device sensor 92, communications interface 99, location sensor 98, and/or user interface 80 and process it to provide data output, for example to indicator 85 and/or to communications interface 99.

In some embodiments, the drug administration device 500 is enclosed in packaging 35. The packaging 35 may further include a combination of a processor 96, memory 97, user interface 80, device indicator 85, device sensor 92, location sensor 98 and/or environment sensors 94 as described herein, and these may be located externally on the housing of the device 500.

A person skilled in the art will appreciate that the universal drug administration device 500 comprising the drug holder 10 and dispensing mechanism 20 can be provided with a variety of the optional features described above, in a number of different combinations. Moreover, the drug administration device 500 can include more than one drug holder 10, optionally with more than one dispensing mechanism 20, such that each drug holder has its own associated dispensing mechanism 20.

### Drug Dosage Forms

Conventionally, drug administration devices utilize a liquid dosage form. It will be appreciated, however that other dosage forms are available.

One such common dosage form is a tablet. The tablet may be formed from a combination of the drug and an excipient that are compressed together. Other dosage forms are pastes, creams, powders, ear drops, and eye drops.

Further examples of drug dosage forms include dermal patches, drug eluting stents and intrauterine devices. In these examples, the body of the device comprises the drug and may be configured to allow the release of the drug under certain circumstances. For example, a dermal patch may comprise a polymeric composition containing the drug. The polymeric composition allows the drug to diffuse out of the polymeric composition and into the skin of the patient. Drug eluting stents and intrauterine devices can operate in an analogous manner. In this way, the patches, stents and intrauterine devices may themselves be considered drug holders with an associated dispensing mechanism.

Any of these dosage forms can be configured to have the drug release initiated by certain conditions. This can allow the drug to be released at a desired time or location after the dosage form has been introduced into the patient. In particular, the drug release may be initiated by an external stimulus. Moreover, these dosage forms can be contained prior to administration in a housing, which may be in the form of packaging. This housing may contain some of the optional features described above which are utilized with the universal drug administration device 500.

The drug administered by the drug administration devices of the present disclosure can be any substance that causes a change in an organism's physiology or psychology when consumed. Examples of drugs that the drug administration devices of the present disclosure can administer include 5-alpha-reductase inhibitors, 5-aminosalicylates, 5HT3 receptor antagonists, ACE inhibitors with calcium channel blocking agents, ACE inhibitors with thiazides, adamantane antivirals, adrenal cortical steroids, adrenal corticosteroid inhibitors, adrenergic bronchodilators, agents for hypertensive emergencies, agents for pulmonary hypertension, aldosterone receptor antagonists, alkylating agents, allergenics, alpha-glucosidase inhibitors, alternative medicines, amebicides, aminoglycosides, aminopenicillins, aminosalicylates, AMPA receptor antagonists, amylin analogs, analgesic combinations, analgesics, androgens and anabolic steroids, Angiotensin Converting Enzyme Inhibitors, angiotensin II inhibitors with calcium channel blockers, angiotensin II inhibitors with thiazides, angiotensin receptor blockers, angiotensin receptor blockers and neprilysin inhibitors, anorectal preparations, anorexiants, antacids, anthelmintics, anti-angiogenic ophthalmic agents, anti-CTLA-4 monoclonal antibodies, anti-infectives, anti-PD-1 monoclonal antibodies, antiadrenergic agents (central) with thiazides, antiadrenergic agents (peripheral) with thiazides, antiadrenergic agents, centrally acting, antiadrenergic agents, peripherally acting, antiandrogens, antianginal agents, antiarrhythmic agents, antiasthmatic combinations, antibiotics/antineoplastics, anticholinergic antiemetics, anticholinergic antiparkinson agents, anticholinergic bronchodilators, anticholinergic chronotropic agents, anticholinergics/antispasmodics, anticoagulant reversal agents, anticoagulants, anticonvulsants, antidepressants, antidiabetic agents, antidiabetic combinations, antidiarrheals, antidiuretic hormones, antidotes, antiemetic/antivertigo agents, antifungals, antigonadotropic agents, antigout agents, antihistamines, antihyperlipidemic agents, antihyperlipidemic combinations, antihypertensive combinations, antihyperuricemic agents, antimalarial agents, antimalarial combinations, antimalarial quinolones, antimanic agents, antimetabolites, antimigraine agents, antineoplastic combinations, antineoplastic detoxifying agents, antineoplastic interferons, antineoplastics, antiparkinson agents, antiplatelet agents, antipseudomonal penicillins, antipsoriatics, antipsychotics, antirheumatics, antiseptic and germicides, antithyroid agents, antitoxins and antivenins, antituberculosis agents, antituberculosis combinations, antitussives, antiviral agents, antiviral boosters, antiviral combinations, antiviral interferons, anxiolytics, sedatives, and hypnotics, aromatase inhibitors, atypical antipsychotics, azole antifungals, bacterial vaccines, barbiturate anticonvulsants, barbiturates, BCR-ABL tyrosine kinase inhibitors, benzodiazepine anticonvulsants, benzodiazepines, beta blockers with calcium channel blockers, beta blockers with thiazides, beta-adrenergic blocking agents, beta-lactamase inhibitors, bile acid sequestrants, biologicals, bisphosphonates, bone morphogenetic proteins, bone resorption inhibitors, bronchodilator combinations, bronchodilators, calcimimetics, calcineurin inhibitors, calcitonin, calcium channel blocking agents, carbamate anticonvulsants, carbapenems, carbapenems/beta-lactamase inhibitors, carbonic anhydrase inhibitor anticonvulsants, carbonic anhydrase inhibitors, cardiac stressing agents, cardioselective beta blockers, cardiovascular agents, catecholamines, cation exchange resins, CD20 monoclonal antibodies, CD30 monoclonal antibodies, CD33 monoclonal antibodies, CD38 monoclonal antibodies, CD52 monoclonal antibodies, CDK 4/6 inhibitors, central nervous system agents, cephalosporins, cephalosporins/beta-lactamase inhibitors, cerumenolytics, CFTR combinations, CFTR potentiators, CGRP inhibitors, chelating agents, chemokine receptor antagonist, chloride channel activators, cholesterol absorption inhibitors, cholinergic agonists, cholinergic muscle stimulants, cholinesterase inhibitors, CNS stimulants, coagulation modifiers, colony stimulating factors, contraceptives, corticotropin, coumarins and indandiones, cox-2 inhibitors, decongestants, dermatological agents, diagnostic radiopharmaceuticals, diarylquinolines, dibenzazepine anticonvulsants, digestive enzymes, dipeptidyl peptidase 4 inhibitors, diuretics, dopaminergic antiparkinsonism agents, drugs used in alcohol dependence, echinocandins, EGFR inhibitors, estrogen receptor antagonists, estrogens, expectorants, factor Xa inhibitors, fatty acid derivative anticonvulsants, fibric acid derivatives, first generation cephalosporins, fourth generation cephalosporins, functional bowel disorder agents, gallstone solubilizing agents, gamma-aminobutyric acid analogs, gamma-aminobutyric acid reuptake inhibitors, gastrointestinal agents, general anesthetics, genitourinary tract agents, GI stimulants, glucocorticoids, glucose elevating agents, glycopeptide antibiotics, glycoprotein platelet inhibitors, glycylcyclines, gonadotropin releasing hormones, gonadotropin-releasing hormone antagonists, gonadotropins, group I antiarrhythmics, group II antiarrhythmics, group III antiarrhythmics, group IV antiarrhythmics, group V antiarrhythmics, growth hormone receptor blockers, growth hormones, guanylate cyclase-C agonists, H. pylori eradication agents, H2 antagonists, hedgehog pathway inhibitors, hematopoietic stem cell mobilizer, heparin antagonists, heparins, HER2 inhibitors, herbal products, histone deacetylase inhibitors, hormones, hormones/antineoplastics, hydantoin anticonvulsants, hydrazide derivatives, illicit (street) drugs, immune globulins, immunologic agents, immunostimulants, immunosuppressive agents, impotence agents, in vivo diagnostic biologicals, incretin mimetics, inhaled anti-infectives, inhaled corticosteroids, inotropic agents, insulin, insulin-like growth factors, integrase strand transfer inhibitor, interferons, interleukin inhibitors, interleukins, intravenous nutritional products, iodinated contrast media, ionic iodinated contrast media, iron products, ketolides, laxatives, leprostatics, leukotriene modifiers, lincomycin derivatives, local injectable anesthetics, local injectable anesthetics with corticosteroids, loop diuretics, lung surfactants, lymphatic staining agents, lysosomal enzymes, macrolide derivatives, macrolides, magnetic resonance imaging contrast media, mast cell stabilizers, medical gas, meglitinides, metabolic agents, methylxanthines, mineralocorticoids, minerals and electrolytes, miscellaneous agents, miscellaneous analgesics, miscellaneous antibiotics, miscellaneous anticonvulsants, miscellaneous antidepressants, miscellaneous antidiabetic agents, miscellaneous antiemetics, miscellaneous antifungals, miscellaneous antihyperlipidemic agents, miscellaneous antihypertensive combinations, miscellaneous antimalarials, miscellaneous antineoplastics, miscellaneous antiparkinson agents, miscellaneous antipsychotic agents, miscellaneous antituberculosis agents, miscellaneous antivirals, miscellaneous anxiolytics, sedatives and hypnotics, miscellaneous bone resorption inhibitors, miscellaneous cardiovascular agents, miscellaneous central nervous system agents, miscellaneous coagulation modifiers, miscellaneous diagnostic dyes, miscellaneous diuretics, miscellaneous genitourinary tract agents, miscellaneous GI agents, miscellaneous hormones, miscellaneous metabolic agents, miscellaneous ophthalmic agents, miscellaneous otic agents, miscellaneous respiratory agents, miscellaneous sex hormones, miscellaneous topical agents, miscellaneous uncategorized agents, miscellaneous vaginal agents, mitotic inhibitors, monoamine oxidase inhibitors, mouth and throat products, mTOR inhibitors, mucolytics, multikinase inhibitors, muscle relaxants, mydriatics, narcotic analgesic combinations, narcotic analgesics, nasal anti-infectives, nasal antihistamines and decongestants, nasal lubricants and irrigations, nasal preparations, nasal steroids, natural penicillins, neprilysin inhibitors, neuraminidase inhibitors, neuromuscular blocking agents, neuronal potassium channel openers, next generation cephalosporins, nicotinic acid derivatives, NK1 receptor antagonists, NNRTIs, non-cardioselective beta blockers, non-iodinated contrast media, non-ionic iodinated contrast media, non-sulfonylureas, Nonsteroidal anti-inflammatory drugs, NS5A inhibitors, nucleoside reverse transcriptase inhibitors (NRTIs), nutraceutical products, nutritional products, ophthalmic anesthetics, ophthalmic anti-infectives, ophthalmic anti-inflammatory agents, ophthalmic antihistamines and decongestants, ophthalmic diagnostic agents, ophthalmic glaucoma agents, ophthalmic lubricants and irrigations, ophthalmic preparations, ophthalmic steroids, ophthalmic steroids with anti-infectives, ophthalmic surgical agents, oral nutritional supplements, other immunostimulants, other immunosuppressants, otic anesthetics, otic anti-infectives, otic preparations, otic steroids, otic steroids with anti-infectives, oxazolidinedione anticonvulsants, oxazolidinone antibiotics, parathyroid hormone and analogs, PARP inhibitors, PCSK9 inhibitors, penicillinase resistant penicillins, penicillins, peripheral opioid receptor antagonists, peripheral opioid receptor mixed agonists/antagonists, peripheral vasodilators, peripherally acting antiobesity agents, phenothiazine antiemetics, phenothiazine antipsychotics, phenylpiperazine antidepressants, phosphate binders, PI3K inhibitors, plasma expanders, platelet aggregation inhibitors, platelet-stimulating agents, polyenes, potassium sparing diuretics with thiazides, potassium-sparing diuretics, probiotics, progesterone receptor modulators, progestins, prolactin inhibitors, prostaglandin D2 antagonists, protease inhibitors, protease-activated receptor-1 antagonists, proteasome inhibitors, proton pump inhibitors, psoralens, psychotherapeutic agents, psychotherapeutic combinations, purine nucleosides, pyrrolidine anticonvulsants, quinolones, radiocontrast agents, radiologic adjuncts, radiologic agents, radiologic conjugating agents, radiopharmaceuticals, recombinant human erythropoietins, renin inhibitors, respiratory agents, respiratory inhalant products, rifamycin derivatives, salicylates, sclerosing agents, second generation cephalosporins, selective estrogen receptor modulators, selective immunosuppressants, selective phosphodiesterase-4 inhibitors, selective serotonin reuptake inhibitors, serotonin-norepinephrine reuptake inhibitors, serotoninergic neuroenteric modulators, sex hormone combinations, sex hormones, SGLT-2 inhibitors, skeletal muscle relaxant combinations, skeletal muscle relaxants, smoking cessation agents, somatostatin and somatostatin analogs, spermicides, statins, sterile irrigating solutions, streptogramins, streptomyces derivatives, succinimide anticonvulsants, sulfonamides, sulfonylureas, synthetic ovulation stimulants, tetracyclic antidepressants, tetracyclines, therapeutic radiopharmaceuticals, therapeutic vaccines, thiazide diuretics, thiazolidinediones, thioxanthenes, third generation cephalosporins, thrombin inhibitors, thrombolytics, thyroid drugs, TNF alfa inhibitors, tocolytic agents, topical acne agents, topical agents, topical allergy diagnostic agents, topical anesthetics, topical anti-infectives, topical anti-rosacea agents, topical antibiotics, topical antifungals, topical antihistamines, topical antineoplastics, topical antipsoriatics, topical antivirals, topical astringents, topical debriding agents, topical depigmenting agents, topical emollients, topical keratolytics, topical non-steroidal anti-inflammatories, topical photochemotherapeutics, topical rubefacient, topical steroids, topical steroids with anti-infectives, transthyretin stabilizers, triazine anticonvulsants, tricyclic antidepressants, trifunctional monoclonal antibodies, ultrasound contrast media, upper respiratory combinations, urea anticonvulsants, urea cycle disorder agents, urinary anti-infectives, urinary antispasmodics, urinary pH modifiers, uterotonic agents, vaccine combinations, vaginal anti-infectives, vaginal preparations, vasodilators, vasopressin antagonists, vasopressors, VEGF/VEGFR inhibitors, viral vaccines, viscosupplementation agents, vitamin and mineral combinations, vitamins, or VMAT2 inhibitors. The drug administration devices of the present disclosure may administer a drug selected from epinephrine, Rebif, Enbrel, Aranesp, atropine, pralidoxime chloride, diazepam, insulin, antropine sulfate, avibactam sodium, bendamustine hydrochloride, carboplatin, daptomycin, epinephrine, levetiracetam, oxaliplatin, paclitaxel, pantoprazole sodium, treprostinil, vasopressin, voriconazole, zoledronic acid, mometasone, fluticasone, ciclesonide, budesonide, beclomethasone, vilanterol, salmeterol, formoterol, umeclidinium, glycopyrrolate, tiotropium, aclidinium, indacaterol, salmeterol, and olodaterol.

As mentioned above, any of a variety of drugs can be delivered using a drug administration device. Examples of drugs that can be delivered using a drug administration device as described herein include Remicade® (infliximab), Stelara® (ustekinumab), Simponi® (golimumab), Simponi Aria® (golimumab), Darzalex® (daratumumab), Tremfya® (guselkumab), Eprex® (epoetin alfa), Risperdal Constra® (risperidone), Invega Sustenna® (paliperidone palmitate), Spravato® (esketamine), ketamine, and Invega Trinza® (paliperidone palmitate).

### Drug Housing

As described above, a dosage form can be provided in a holder that is appropriate for the particular dosage form being utilized. For example, a drug in a liquid dosage form can be held prior to administration within a holder in the form of a vial with a stopper, or a syringe with a plunger. A drug in solid or powder dosage form, e.g., as tablets, may be contained in a housing which is arranged to hold the tablets securely prior to administration.

The housing may comprise one or a plurality of drug holders, where each holder contains a dosage form, e.g., the drug can be in a tablet dosage form and the housing can be in the form of a blister pack, where a tablet is held within each of a plurality of holders. The holders being in the form of recesses in the blister pack.

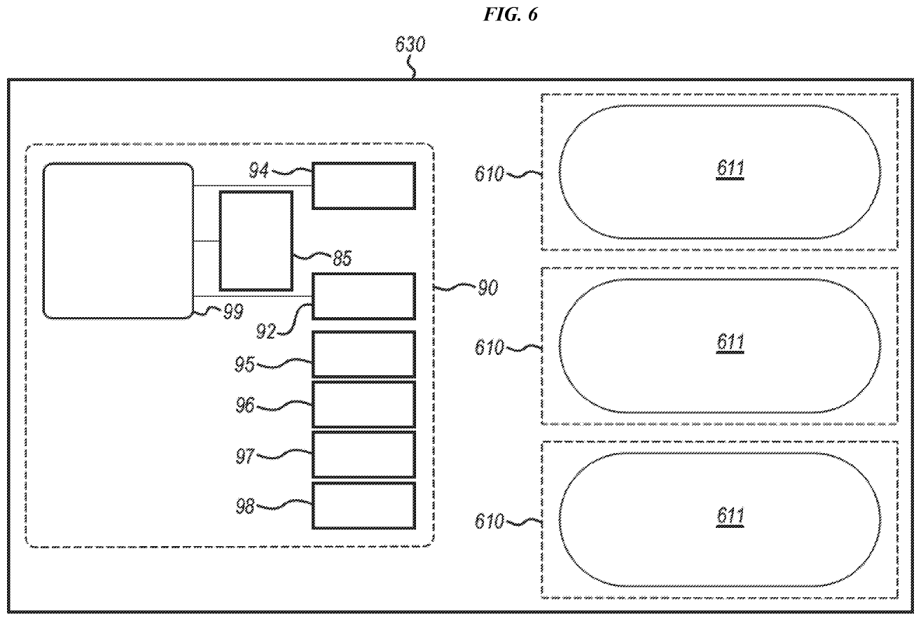

FIG. 6 depicts a housing 630 that comprises a plurality of drug holders 610 that each contain a dosage form 611. The housing 630 may have at least one environment sensor 94, which is configured to sense information relating to the environment in which the housing 630 is present, such as the temperature of the environment, time or location. The housing 630 may include at least one device sensor 92, which is configured to sense information relating to the drug of the dosage form 611 contained within the holder 610. There may be a dedicated location sensor 98 which is configured to determine the geographical location of the housing 630, e.g., via satellite position determination, such as GPS.

The housing 630 may include an indicator 85 which is configured to present information about the status of the drug of the dosage form 611 contained within the holder 610 to a user of the drug housing. The housing 630 may also include a communications interface 99 which can communicate information externally via a wired or wireless transfer of data pertaining to the drug housing 630, environment, time or location and/or the drug itself.

If required, the housing 630 may comprise a power supply 95 for delivering electrical power to one or more electrical components of the housing 630. The power supply 95 can be a source of power which is integral to housing 630 and/or a mechanism for connecting the housing 630 to an external source of power. The housing 630 may also include a device computer system 90 including processor 96 and memory 97 powered by the power supply 95 and in communication with each other, and optionally with other electrical and control components of the housing 630, such as the environment sensor 94, location sensor 98, device sensor 92, communications interface 99, and/or indicator 85. The processor 96 is configured to obtain data acquired from the environment sensor 94, device sensor 92, communications interface 99, location sensor 98, and/or user interface 80 and process it to provide data output, for example to indicator 85 and/or to communications interface 99.

The housing 630 can be in the form of packaging. Alternatively, additional packaging may be present to contain and surround the housing 630.

The holder 610 or the additional packaging may themselves comprise one or more of the device sensor 92, the environment sensor 94, the indicator 85, the communications interface 99, the power supply 95, location sensor 98, and device computer system including the processor 96 and the memory 85, as described above.

### Electronic Communication

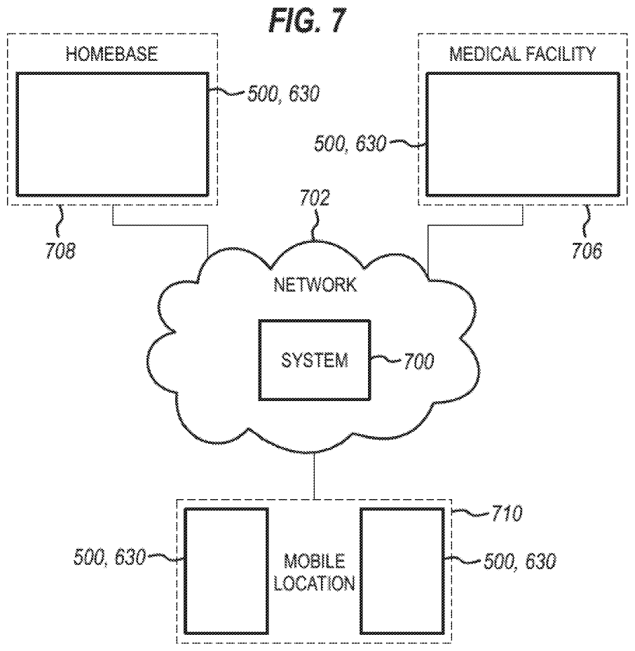

As mentioned above, communications interface 99 may be associated with the drug administration device 500 or drug housing 630, by being included within or on the housing 30, 630, or alternatively within or on the packaging 35. Such a communications interface 99 can be configured to communicate with a remote computer system, such as central computer system 700 shown in FIG. 7. As shown in FIG. 7, the communications interface 99 associated with drug administration device 500 or housing 630 is configured to communicate with a central computer system 700 through a communications network 702 from any number of locations such as a medical facility 706, e.g., a hospital or other medical care center, a home base 708 (e.g., a patient's home or office or a care taker's home or office), or a mobile location 710. The communications interface 99 can be configured to access the system 700 through a wired and/or wireless connection to the network 702. In an exemplary embodiment, the communications interface 99 of FIG. 6 is configured to access the system 700 wirelessly, e.g., through Wi-Fi connection(s), which can facilitate accessibility of the system 700 from almost any location in the world.

A person skilled in the art will appreciate that the system 700 can include security features such that the aspects of the system 700 available to any particular user can be determined based on, e.g., the identity of the user and/or the location from which the user is accessing the system. To that end, each user can have a unique username, password, biometric data, and/or other security credentials to facilitate access to the system 700. The received security parameter information can be checked against a database of authorized users to determine whether the user is authorized and to what extent the user is permitted to interact with the system, view information stored in the system, and so forth.

### Computer System

As discussed herein, one or more aspects or features of the subject matter described herein, for example components of the central computer system 700, processor 96, power supply 95, memory 97, communications interface 99, user interface 80, device indicators 85, device sensors 92, environment sensors 94 and location sensors 98, can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computer system may include clients and servers. A client and server are generally remote from each other and typically interact through a communications network, e.g., the Internet, a wireless wide area network, a local area network, a wide area network, or a wired network. The relationship of client and server arises by virtue of computer programs running on the respective computers and having a client-server relationship to each other.

The computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, one or more aspects or features of the subject matter described herein, for example user interface 80 (which can be integrated or separate to the administration device 500 or housing 630), can be implemented on a computer having a display screen, such as for example a cathode ray tube (CRT) or a liquid crystal display (LCD) or a light emitting diode (LED) monitor for displaying information to the user. The display screen can allow input thereto directly (e.g., as a touch screen) or indirectly (e.g., via an input device such as a keypad or voice recognition hardware and software). Other kinds of devices can be used to provide for interaction with a user as well. For example, feedback provided to the user can be any form of sensory feedback, such as for example visual feedback, auditory feedback, or tactile feedback; and input from the user may be received in any form, including, but not limited to, acoustic, speech, or tactile input. As described above, this feedback may be provided via one or more device indicators 85 in addition to the user interface 80. The device indicators 85 can interact with one or more of device sensor(s) 92, environment sensor(s) 94 and/or location sensor(s) 98 in order to provide this feedback, or to receive input from the user.

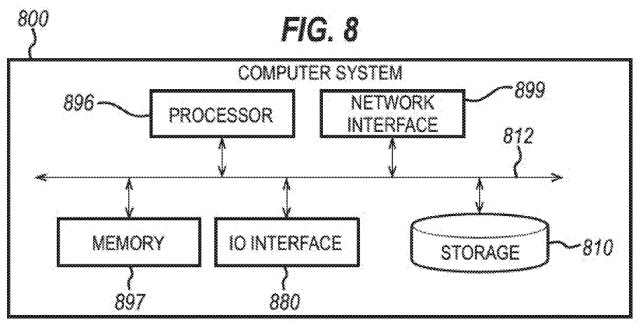

FIG. 8 illustrates one exemplary embodiment of the computer system 700, depicted as computer system 800. The computer system includes one or more processors 896 configured to control the operation of the computer system 800. The processor(s) 896 can include any type of microprocessor or central processing unit (CPU), including programmable general-purpose or special-purpose microprocessors and/or any one of a variety of proprietary or commercially available single or multi-processor systems. The computer system 800 also includes one or more memories 897 configured to provide temporary storage for code to be executed by the processor(s) 896 or for data acquired from one or more users, storage devices, and/or databases. The memory 897 can include read-only memory (ROM), flash memory, one or more varieties of random access memory (RAM) (e.g., static RAM (SRAM), dynamic RAM (DRAM), or synchronous DRAM (SDRAM)), and/or a combination of memory technologies.

The various elements of the computer system are coupled to a bus system 812. The illustrated bus system 812 is an abstraction that represents any one or more separate physical busses, communication lines/interfaces, and/or multi-drop or point-to-point connections, connected by appropriate bridges, adapters, and/or controllers. The computer system 800 also includes one or more network interface(s) 899 (also referred to herein as a communications interface), one or more input/output (TO) interface(s) 880, and one or more storage device(s) 810.

The communications interface(s) 899 are configured to enable the computer system to communicate with remote devices, e.g., other computer systems and/or devices 500 or housings 630, over a network, and can be, for example, remote desktop connection interfaces, Ethernet adapters, and/or other local area network (LAN) adapters. The IO interface(s) 880 include one or more interface components to connect the computer system 800 with other electronic equipment. For example, the IO interface(s) 880 can include high speed data ports, such as universal serial bus (USB) ports, 1394 ports, Wi-Fi, Bluetooth, etc. Additionally, the computer system can be accessible to a human user, and thus the IO interface(s) 880 can include displays, speakers, keyboards, pointing devices, and/or various other video, audio, or alphanumeric interfaces. The storage device(s) 810 include any conventional medium for storing data in a non-volatile and/or non-transient manner. The storage device(s) 810 are thus configured to hold data and/or instructions in a persistent state in which the value(s) are retained despite interruption of power to the computer system. The storage device(s) 810 can include one or more hard disk drives, flash drives, USB drives, optical drives, various media cards, diskettes, compact discs, and/or any combination thereof and can be directly connected to the computer system or remotely connected thereto, such as over a network. In an exemplary embodiment, the storage device(s) 810 include a tangible or non-transitory computer readable medium configured to store data, e.g., a hard disk drive, a flash drive, a USB drive, an optical drive, a media card, a diskette, or a compact disc.

The elements illustrated in FIG. 8 can be some or all of the elements of a single physical machine. In addition, not all of the illustrated elements need to be located on or in the same physical machine.

The computer system 800 can include a web browser for retrieving web pages or other markup language streams, presenting those pages and/or streams (visually, aurally, or otherwise), executing scripts, controls and other code on those pages/streams, accepting user input with respect to those pages/streams (e.g., for purposes of completing input fields), issuing HyperText Transfer Protocol (HTTP) requests with respect to those pages/streams or otherwise (e.g., for submitting to a server information from the completed input fields), and so forth. The web pages or other markup language can be in HyperText Markup Language (HTML) or other conventional forms, including embedded Extensible Markup Language (XML), scripts, controls, and so forth. The computer system 800 can also include a web server for generating and/or delivering the web pages to client computer systems.

As shown in FIG. 7, the computer system 800 of FIG. 8 as described above may form the components of the central computer system 700 which is in communication with one or more of the device computer systems 90 of the one or more individual drug administration devices 500 or housings 630. Data, such as operational data of the devices 500 or housings 630, medical data acquired of patients by such devices 500 or housings 630 can be exchanged between the central and device computer systems 700, 90.

As mentioned the computer system 800 as described above may also form the components of a device computer system 90 which is integrated into or in close proximity to the drug administration device 500 or housing 630. In this regard, the one or more processors 896 correspond to the processor 96, the network interface 799 corresponds to the communications interface 99, the IO interface 880 corresponds to the user interface 80, and the memory 897 corresponds to the memory 97. Moreover, the additional storage 810 may also be present in device computer system 90.

In an exemplary embodiment, the computer system 800 can form the device computer system 90 as a single unit, e.g., contained within a single drug administration device housing 30, contained within a single package 35 for one or more drug administration devices 500, or a housing 630 that comprises a plurality of drug holders 610. The computer system 800 can form the central computer system 700 as a single unit, as a single server, or as a single tower.

The single unit can be modular such that various aspects thereof can be swapped in and out as needed for, e.g., upgrade, replacement, maintenance, etc., without interrupting functionality of any other aspects of the system. The single unit can thus also be scalable with the ability to be added to as additional modules and/or additional functionality of existing modules are desired and/or improved upon.

The computer system can also include any of a variety of other software and/or hardware components, including by way of example, operating systems and database management systems. Although an exemplary computer system is depicted and described herein, it will be appreciated that this is for sake of generality and convenience. In other embodiments, the computer system may differ in architecture and operation from that shown and described here. For examples, the memory 897 and storage device 810 can be integrated together or the communications interface 899 can be omitted if communication with another computer system is not necessary.

### Implementations

### Confirmation of Drug Administration

It can be desirable to monitor compliance with the guidance that is associated with drugs that are administered to a patient in various dosage forms. This compliance monitoring can provide assurance that correct procedures are being followed and avoid adoption of incorrect and potentially dangerous approaches. Further, this can also enable optimization of the administration of the drug to the patient. Various methods, systems, and devices described herein may confirm successful administration of a drug to a patient, which may improve patient safety and compliance by quickly identifying that a problem has occurred when administering the drug.

It can be desirable to monitor the delivery of drugs to identify delivery problems or other issues, particularly in relation to drug trials or conformity with dosing prescriptions. Further, for some drugs, it can be desirable to administer the drug to the patient in an inactive form and to activate the drug at a target location in the body in order to improve efficacy and safety. For example, chemotherapy drugs can be systemically delivered to a patient but activated only at the tumor site so that the chemotherapy drug is effective on tumor cells while harm caused to healthy cells elsewhere in the patient is minimized. Various methods, systems, and devices described herein may improve drug efficacy, as well as safety and compliance, by improving local drug activation.

In an exemplary embodiment, at least one dispensing mechanism parameter can be compared with acceptable dispensing mechanism parameters, and at least one administration parameter can be compared with acceptable administration parameters. These comparisons may give a user of a drug administration device confidence that the drug administration device has operated successfully. Herein, successful administration is used to mean that operation of a dispensing mechanism of the drug administration device is determined to be complete. This confirmation of successful administration may make the drug administration procedure safer for a patient receiving the drug, as the patient, and/or a healthcare professional, can be alerted quickly if successful administration is not confirmed, and can therefore intervene if needed to take a corrective action, e.g., ordering a new drug administration device, repairing the drug administration device, delivering a drug dose using a different drug administration device, increasing a maximum number of dose administrations allowed from the drug administration device by one or more to allow for one or more doses of drug to be delivered from the drug administration device, etc. Confirming administration may prevent incorrect decisions regarding future administrations being made, as the source of a problem can be identified more easily. Confirming administration may reduce wastage of a drug. If some of the drug is not being administered successfully, the patient, and/or a healthcare professional, can be alerted to this unsuccessful administration to allow for adjustment of future administrations to reduce wastage.

In general, an acceptable parameter defines a value (or a range of values) that is indicative of successful delivery of a drug from a drug administration device. The acceptable parameter can be predefined prior to use of the drug administration device, such as by being established by a manufacturer of the drug administration device and/or the drug being delivered by the drug administration device. For example, an acceptable parameter can include a speed of a drug administration device's needle being inserted into a patient when the drug administration device is an injector that includes a needle. Too slow a speed can be indicative of failed needle insertion and thus failed drug delivery. For another example, an acceptable parameter can include an angular orientation of an injection device relative to a patient. The injection device not being at a proper angular orientation relative to the patient during drug delivery can indicate that the ejection of the drug from the injection device is not likely to have resulted in all the drug having been properly injected into the patient. The proper angular orientation of an injection device can be a vertical, substantially perpendicular orientation relative to the patient's skin versus an improper position of being at a non-perpendicular angle relative to the patient's skin. A person skilled in the art will appreciate that the angle may not be precisely perpendicular (precisely 90°) but nevertheless be considered to be substantially perpendicular for any of a variety of reasons, such as manufacturing tolerance and sensitivity of measurement equipment. For yet another example, an acceptable parameter can include motion of a drug canister of an inhaler. Too little vertical motion of the drug canister downward can be indicative of drug not being expelled properly or at all. For still another example, an acceptable parameter can include motion of a dispensing head of a nasal spray device that is configured to be pushed downward to cause drug delivery through an opening in the dispensing head. Too little vertical motion of the dispensing head downward can be indicative of drug not being expelled properly or at all. For another example, an acceptable parameter can include an angular orientation of a nasal spray device relative to a patient. The nasal spray device not being at a proper angular orientation relative to the patient during drug delivery can indicate that the spray of the drug into the patient's nostril (or both of the patient's nostrils for a dual-spray nasal spray device) is not likely to have been properly disseminated in the patient's nasal cavity. The proper angular orientation of a nasal spray device can be in a range of 30° to 60°. For yet another example, an acceptable parameter can relate to a motor of a drug administration device, such as a speed of the motor or a duration of operation of the motor. Too slow a speed can be indicative of failed drug delivery or a failed attempt at motor-driven drug mixing in the drug administration device prior to drug delivery. Too short a duration of operation of the motor can be indicative of failed drug delivery or a failed attempt at motor-driven drug mixing in the drug administration device prior to drug delivery. For another example, an acceptable parameter can include a flow rate of a drug administered by a drug administration device. Too low a flow rate can be indicative of failed drug delivery. For another example, an acceptable parameter can include an amount of liquid present in a vicinity of an injection site. Too much liquid on the patient's skin surface can be indicative of failed drug delivery when the drug is a liquid. For another example, an acceptable parameter can include a heart rate of the patient. Too high a heart rate may be indicative of an incorrect drug dose being administered, e.g., too much of the drug was administered. For another example, an acceptable parameter can include a blood pressure of the patient. Too high a blood pressure may be indicative of an incorrect drug dose being administered, e.g., too much of the drug was administered.

In an exemplary embodiment, the comparison of a parameter, e.g., a dispensing mechanism parameter or an administration parameter, to an acceptable parameter is performed by a processor, such as a processor of the drug administration device or of an external device external to and in electronic communication with the drug administration device. The comparison of a parameter (e.g., a measured dispensing mechanism parameter or a measured administration parameter) to an acceptable parameter can be performed in a variety of ways. For example, an acceptable parameter can include a predefined range of values, and the comparing can include determining whether a measured parameter is within the predefined range of values so as to be indicative of successful drug delivery. For another example, the acceptable parameter can include a predefined threshold value, and the comparing can include determining whether a measured parameter is above the predefined threshold value so as to be indicative of successful drug delivery. For another example, the acceptable parameter can include a predefined threshold value, and the comparing can include determining whether a measured parameter is below the predefined threshold value so as to be indicative of successful drug delivery.

The drug administration device can be any drug administration device described herein. The drug administration device can be one that effects the administration of drug automatically, e.g., without a manual user input. Alternatively, the drug administration device can require a manual user input in order to initiate administration.

The at least one dispensing mechanism parameter can be any parameter associated with dispensing of the drug from the drug administration device. In general, a dispensing mechanism parameter is a characteristic of operation of a dispensing mechanism of the drug administration device. In this way, the dispensing mechanism parameter provides an indication that the operation of the dispensing mechanism is complete, e.g., the operation of the dispensing mechanism has occurred to the intended extent. A plurality of dispensing mechanism parameters can be measured and utilized in determining whether the operation of the dispensing mechanism is complete. Measuring and comparing a plurality of dispensing mechanism parameters may result in a more accurate assessment of whether the dispensing mechanism operation is complete than if only one dispensing mechanism parameter is measured and compared. The plurality of dispensing mechanism parameters that are measured can include any plural number of the dispensing mechanism parameters described herein.

Measuring at least one dispensing mechanism parameter provides an initial indication that the drug has been successfully dispensed by the drug administration device. As noted above, the measured dispensing mechanism parameter is compared with acceptable dispensing mechanism parameters. The acceptable dispensing mechanism parameters may be stored in a memory, e.g., a memory of the drug administration device and/or a memory of an external device external to and in electronic communication with the drug administration device. The acceptable dispensing mechanism parameters can be predefined as the dispensing mechanism parameters known to represent completion of dispensing mechanism operation, as discussed above.

The at least one administration parameter can be any parameter associated with administration of the drug. In general, an administration parameter is a characteristic of the administration of the drug from the drug administration device. The at least one administration parameter is distinct from the at least one dispensing mechanism parameter. The at least one administration parameter can be measured simultaneously to the at least one dispensing mechanism parameter. The at least one administration parameter can be measured after the at least one dispensing mechanism parameter. Measuring the at least one administration parameter allows confirmation that the drug has been successfully administered and acts as an independent check on the confirmation that the operation of the dispensing mechanism is complete. Measuring the at least one administration parameter may increase the chance of detecting unsuccessful administration, as compared to only measuring the dispensing mechanism parameter(s), and thereby allow quicker intervention to prevent the patient being harmed.

A plurality of administration parameters can be measured and utilized when confirming whether administration was successful. Measuring and comparing a plurality of administration parameters may result in a more accurate assessment of whether the administration was successful, since more checks are performed than when only one administration parameter is measured and compared. The plurality of administration parameters that are measured can include any plural number of the administration parameters described herein.

The acceptable administration parameters can be predefined, as discussed above, and can be stored in a memory of the drug administration device and/or a memory of an external device external to and in electronic communication with the drug administration device. In at least some embodiments, the acceptable administration parameters can be calculated using the measured dispensing mechanism parameter(s). This calculation can be conducted by a processor, such as a processor of the drug administration device or of an external device external to and in electronic communication with the drug administration device. This calculation allows for a variation in acceptable administration parameters depending on a characteristic of the operation of the dispensing mechanism. This calculation accounts for the possibility that the acceptable administration parameters may be dictated by the particular operation of the dispensing mechanism. For example, an increased volume of drug administered to a patient may be associated with an expectation that the patient's physiological response will increase. Accordingly, where this physiological response is the measured administration parameter, the acceptable administration parameters will need to be adjusted to account for the increased drug volume. In such as case, a predefined baseline of the administration parameter can be established as the acceptable administration parameter as discussed above, and this baseline can be adjusted in view of the relevant measured dispensing mechanism parameter(s).

A user of the drug administration device can be notified of an unsuccessful administration as determined by the comparison of the at least one dispensing mechanism parameter with acceptable dispensing mechanism parameters and the comparison of the at least one administration parameter with acceptable administration parameters. Alternatively, or in addition, the user can be notified of a successful administration as determined by the comparison of the at least one dispensing mechanism parameter with acceptable dispensing mechanism parameters and the comparison of the at least one administration parameter with acceptable administration parameters. The notification can be particular to whether administration was successful. The notification can be effected by a device indicator.

Further operation of the drug administration device can be modified based on the at least one dispensing mechanism parameter and/or the at least one administration parameter. Enabling modification of further operation of the drug administration device allows adjustments to be made that may make it more likely for further administrations to be successful. These modifications may also reduce further wastage of the administered drug.

The drug administration device can be configured to effect the modification automatically, e.g., without requiring user input. Alternatively, the drug administration device can be configured to prompt the user to manually effect the modification. The drug administration device can be configured to prompt the user via a device indicator and/or a user interface. The required modification can be determined based on the measured dispensing mechanism parameter(s) and/or the measured administration parameter(s). A look-up table can be stored in a memory associated with the device (either on-board or off-board the drug administration device) that defines the operational change required for given parameters. The change can then be automatically effected by the device, or the user may be prompted to make the required change based on the operational change indicated in the look-up table.

Modifying the further operation of the drug administration device can include preventing the further operation of the drug administration device when successful administration was not confirmed. This prevention of further operation prevents further unsuccessful administrations and can allow the user to be prompted to address the problem that caused the administration to be unsuccessful before further operation of the drug administration device. Preventing the further operation of the drug administration device can include any method of stopping further administration of a drug. For example, preventing the further operation of the drug administration device can include disabling a power supply of the drug administration device, in particular disabling the power supply to the dispensing mechanism of the drug administration device. Disabling the power supply can include, e.g., a processor being configured to open or close a switch that when closed allows the power supply to supply power and when open prevents the power supply from supplying power. For another example, preventing the further operation of the drug administration device can include enabling a device operation prevention mechanism. For yet another example, the drug administration device can be prevented from delivering a subsequent dose of the drug by changing at least one variable parameter of an algorithm, thus resulting effectively in the subsequent dose being equivalent to zero drug being administered. The algorithm can be stored on the drug administration device, e.g., in a memory thereof, and can be executable on board the drug administration device, e.g., by a processor thereof, to administer a dose of the drug from the drug administration device to a patient. The algorithm is stored in the form of one or more sets of pluralities of data points defining and/or representing instructions, notifications, signals, etc. to control functions of the device and administration of the drug. The at least one variable parameter is among the algorithm's data points, e.g., are included in instructions for drug delivery, and are thus each able to be changed by changing one or more of the stored pluralities of data points of the algorithm. After the at least one variable parameter has been changed, subsequent execution of the algorithm administers another dose of the drug according to the changed algorithm. As such, drug delivery over time can be managed for a patient to increase the beneficial results of the drug by taking into consideration actual situations of the patient and actual results of the patient receiving doses of the drug. The at least one variable parameter that is changed to effectively result in the subsequent dose being equivalent to zero drug being administered can be, e.g. changing a dose amount variable parameter to zero, by changing a dose frequency variable parameter to a never-achievable time period, and/or by changing a maximum number of remaining device actuations variable parameter to zero.

Modifying the further operation of the drug administration device can include modifying a dosage volume to be administered during further operation of the drug administration device. This modification of dosage volume enables the dosage volume to be increased or decreased if the at least one administration parameter indicates that the administered dosage volume was too low or too high. This modification of dosage volume prevents the user of the drug administration device suffering adverse effects associated with a dosage that is too low or too high. A processor, e.g., a processor of the drug administration device or a processor located remote to, located outside of, and in electronic communication with the drug administration device, may calculate the change in dosage volume. The drug administration device can be configured to automatically update the dosage volume. The drug administration device can be configured to require input from the user, e.g., via a user interface, to confirm that the newly calculated dosage volume should be used for subsequent administrations. Modifying the dosage volume can include, for example, changing at least one variable parameter of the algorithm that defines dosage volume either by increasing or decreasing the value of the parameter.

Modifying the further operation of the drug administration device can include modifying a frequency with which the drug is administered by the drug administration device. This frequency modification enables intervals at which the drug is administered to be altered. This interval alteration may be desirable if the at least one administration parameter indicates that the drug is being administered either too frequently or too infrequently, which risks harming the patient. This interval alteration may be desirable to change to a never-achievable frequency if the at least one dispensing mechanism parameter indicates that successful administration was not achieved, which may be indicative of a device malfunction that requires repair of the drug administration device before further use or that the drug administration device can no longer be effectively used to deliver drug. A processor, e.g., a processor of the drug administration device or a processor located remote to, located outside of, and in electronic communication with the drug administration device, can be configured to calculate the change in frequency. The drug administration device can be configured to automatically update the frequency. The drug administration device can be configured to require input from the user, e.g., via a user interface, to confirm that the newly calculated frequency should be utilized for subsequent administrations. Modifying the frequency can include, for example, changing at least one variable parameter of the algorithm that defines frequency with which the drug is administered by the drug administration device either by increasing or decreasing the value of the parameter.

Modifying the further operation of the drug administration device can include modifying a rate at which the drug is administered by the drug administration device. In other words, the rate at which the drug is dispensed from the drug administration device during an administering event can be modified. This rate modification enables the time for dispensing the drug to be altered. This rate modification may be desirable if the at least one administration parameter indicates that the drug is being administered too quickly or too slowly, which risks harm to the patient. A processor, e.g., a processor of the drug administration device or a processor located remote to, located outside of, and in electronic communication with the drug administration device, can be configured to calculate the change in rate. The drug administration device can be configured to automatically update the rate. The drug administration device can be configured to require input from the user, e.g., via a user interface, to confirm that the newly calculated rate should be utilized for subsequent administrations. Modifying the rate can include, for example, changing at least one variable parameter of the algorithm that defines rate at which the drug is administered by the drug administration device either by increasing or decreasing the value of the parameter. For another example, modifying the rate can include changing a speed at which a motor drives delivery of the drug from the drug administration device, with a speed decrease corresponding to a reduction of the rate and a speed increase corresponding to an increase of the rate.

A user can be notified that the further operation of the drug administration device has been modified. This notification enables the user to remain informed of changes to the operation of the drug administration device so that the user can check that they approve of the modification(s) to the operation of the drug administration device. This notification is especially relevant for when the drug administration device automatically performs the modification.

Notifying the user that the further operation of the drug administration device has been modified can be effected by a device indicator. The notification can include one or more of a visual feedback, an auditory feedback, and a tactile feedback. The notification enables the user to be easily alerted to any modifications made to further operation of the drug administration device. The visual feedback can be provided using an LED. The LED can be configured to flash to indicate that the further operation of the drug administration device has been modified. The LED can be configured to flash at a different rate or, a different color LED may flash, depending on the modification made. The visual feedback can be provided via a display screen of a computer system. The auditory feedback can include a series of beeps. The beeps can vary depending on the modification made. The tactile feedback can include the drug administration device vibrating. A frequency or magnitude of the vibrations can vary depending on the modifications made.

When the dispensing mechanism comprises a motor, the at least one dispensing mechanism parameter can include a characteristic of operation of the motor. For example, the at least one dispensing mechanism parameter can be a speed at which the motor operates, power drawn by the motor, and/or a duration for which the motor operates. The motor can be any motor capable of operating the dispensing mechanism. The at least one administration parameter can include a characteristic of the operation of the motor as detailed in relation to the at least one dispensing mechanism parameter, with the proviso that the administration parameter(s) utilized are distinct from the dispensing mechanism parameter(s).

When the dispensing mechanism comprises a displaceable component, the at least one dispensing mechanism parameter can include a characteristic of the displaceable component. Operation of the dispensing mechanism can include displacing the displaceable component from a first position to a second position. For example, the at least one dispensing mechanism parameter can include a distance by which the displaceable component has moved, a speed with which the displaceable component has moved, and/or acceleration of the displaceable component. The at least one administration parameter can include a characteristic of the operation of the displaceable component as detailed in relation to the at least one dispensing mechanism parameter, with the proviso that the administration parameter(s) utilized are distinct from the dispensing mechanism parameter(s). Examples of displaceable components include a needle of an infusion pump or an injection device that moves into a patient; a spring of an infusion pump or an injection device that moves to cause a needle of the injection device to move into a patient; a needle shield or other dispensing mechanism protection mechanism of an injection device that slides into a housing of the injection device to provide access to the injection device's discharge nozzle; a spring of a nasal spray device that moves to cause drug to be released from a drug holder of the nasal spray device and ejected through a nozzle of the nasal spray device; a trigger or other triggering mechanism of an injection device, infusion pump, inhaler, or nasal spray device; a drive element of an injection device; a dispensing head of a nasal spray device that is configured to be pushed downward to cause drug delivery through an opening in the dispensing head; a valve of an inhaler; and a drug canister or other drug holder of an inhaler that moves during drug delivery.

The displacement of the displaceable component can be measured using a Hall effect sensor. A Hall effect sensor provides a reliable measurement of the displacement since a Hall effect sensor is not affected by the presence of dust particles, or other physical objects, which can obscure a line of sight of other sensors and thus affect the measurements. The displacement of the displaceable component can be measured instead or additionally using, e.g., a motion sensor and/or a pressure sensor.

The at least one dispensing mechanism parameter can include a characteristic of movement of the drug. For example, the at least one dispensing mechanism parameter can include a flow rate of the drug administered by the drug administration device, which may enable a total volume of drug administered to be calculated and can therefore be used to confirm operation of the device. The flow rate can be measured by a volumetric flow meter. The flow rate can be measured by a piston meter. The flow rate can be measured by an oval gear meter. The flow rate can be measured by a pressure-based meter. The flow rate can be measured by a Venturi meter. The flow rate can be measured in a vicinity of an outlet of the drug administration device. The vicinity of the outlet of the drug administration device generally refers to an area near the outlet but not directly at the outlet to provide flow rate data that is substantially the same as the flow rate of the drug at the outlet without having to provide any sensor(s) and/or other measurement mechanisms too close to the outlet so as to possibly interfere with drug flowing therethrough. The flow rate can be measured at the outlet of the drug administration device. The at least one administration parameter can include a characteristic of movement of the drug as detailed in relation to the at least one dispensing mechanism parameter, with the proviso that the administration parameter(s) utilized are distinct from the dispensing mechanism parameter(s).

The at least one administration parameter can include a characteristic relating to a drug administration site on the patient, e.g., an area of the patient that receives the drug, such as the area of the patient around an injection site. By monitoring the drug administration site on the patient, changes associated with a successful administration or an unsuccessful administration can be used to determine whether a particular administration was successful. For example, measuring the at least one administration parameter can include determining an amount of liquid present in a vicinity of the injection site. The vicinity of the injection site generally refers to an area near the injection site but not directly at the injection site to provide information indicative of the injection site without having to provide any sensor(s) and/or other measurement mechanisms too close to the injection site so as to possibly interfere with drug injection at the injection site.

The liquid measurement can be performed using any method suitable for determining an amount of liquid at a location. Measuring the amount of liquid present in the vicinity of an injection site is a check for whether a liquid drug has been successfully administered into the patient or if, instead, the liquid drug has merely been deposited on a surface of the patient such as may occur in the event of leakage and/or wastage of the drug. This liquid measurement may therefore indicate a potential administration problem. Determining the amount of liquid present in the vicinity of an injection site can be done by a liquid detection sensor. Determining the amount of liquid present in the vicinity of an injection site can be done by a moisture sensor.

The at least one administration parameter can include an angular orientation of the drug administration device. Angular orientation can be measured using, e.g., an accelerometer, a gyro, a tilt/angle switch (mercury free), a position sensor, etc. As mentioned above, some drug administration devices should be at a particular angular orientation relative to the patient during drug administration to help ensure that the drug is delivered properly.

The at least one administration parameter can include a physiological parameter of a user of the drug administration device, e.g., a characteristic of the physiology of the patient. Measuring the at least one physiological parameter may enable confirmation that the drug has been successfully administered, as the drug is having a physiological effect on the user. This confirmation of successful administration may make the drug administration procedure safer for the patient, as the patient, and/or a healthcare professional, can be alerted quickly if successful administration is not confirmed, and can therefore intervene quickly if needed. The physiological parameter can be any physiological parameter of a user that will vary upon administration of a drug such that it can be used to confirm successful administration. The physiological parameter can be a heart rate of the user. The heart rate of the user can be measured using, e.g., a heart rate monitor. The physiological parameter can be blood pressure of the user. The blood pressure of the user can be measured using, e.g., a sphygmomanometer or a blood pressure monitor.

Alternatively, or in addition, to the at least one administration parameter being measured and including a physiological parameter of a user of the drug administration device, at least one physiological parameter can be measured via the at least one dispensing mechanism parameter during a successful administration of the drug. For example, when the at least one dispensing mechanism parameter includes a flow rate of a drug, the measured flow rate can be used to determine the at least one physiological parameter. For example, periodic variations in the flow rate are indicative of a heart rate of the user. Detecting the heart rate via the flow rate is an indication of an intact connection of the drug administration device to a vein of the patient. Absence of a characteristic variation in the flow rate caused by the heart rate indicates an interrupted connection between the vein and the drug administration device. Being able to determine the heart rate therefore is an indicator of successful administration.

Further operation of the drug administration device can be modified based on the at least one dispensing mechanism parameter and/or the at least one physiological parameter. Enabling modification of further operation of the drug administration device allows adjustments to be made that may make it more likely for further administrations to be successful, as discussed herein.

An operational status of the drug administration device can be assessed before operation of the dispensing mechanism. This assessment can include assessing any feature of the drug administration device that is required for successful administration of the drug. This assessment enables the user to have confidence that the drug administration device will successfully administer the drug, as the dispensing mechanism's operational status has been assessed.

Assessing the operational status of the drug administration device can include analyzing a power supply of the drug administration device to verify that the power supply has sufficient charge for successful administration. A sufficient charge can be a predefined minimum charge needed for successful administration. The predefined minimum charge can be stored in a memory for access by a processor performing the analysis. This analysis of the power supply confirms whether there is sufficient charge for successful administrations and therefore narrows down the potential reasons for failure, should the administration be unsuccessful.

Assessing the operational status of the drug administration device can also, or alternatively, include moving the displaceable component of the drug administration device a predefined distance. By confirming completion of this movement it is possible to confirm that the displaceable component is functional and therefore narrows down potential reasons for failure, should the administration be unsuccessful.

Operation of the drug administration device can be prevented if assessing the operational status of the drug administration device indicates that administration would not be successful. This prevention would prevent an administration from only partially completing, which may harm the patient. Preventing operation of the drug administration device can include any of the methods described herein. For example, preventing the operation of the drug administration device can include disabling a power supply of a motor of the drug administration device. For another example, preventing the operation of the drug administration device can include enabling a device operation prevention mechanism. For yet another example, preventing the operation of the drug administration device can include changing at least one variable parameter of an algorithm used in controlling drug administration from the drug administration device.

A user of the drug administration device can be notified that operation of the drug administration device is being prevented. Notifying the user can include any of the methods of notifying a user described herein. In particular, the notification can include one or more of a visual feedback, an auditory feedback, and a tactile feedback.

The user can be notified whether the administration was successful. This notification provides reassurance to the user that the administration was successful and alerts the user if any action is required. Notifying the user whether the administration was successful can include any of the methods of notifying a user described herein. In particular, the notification can include one or more of a visual feedback, an auditory feedback, and a tactile feedback.

A sensor can be configured to measure the at least one dispensing mechanism parameter. Such a sensor is also referred to herein as a “dispensing sensor.” One or more dispensing sensors can be used. This measurement generates dispensing mechanism data, which allows the drug administration system or drug administration device to determine whether the drug has been successfully dispensed by the drug administration device. The dispensing mechanism data corresponding to each of the dispensing mechanism parameters can be used for making the comparisons described herein with respect to comparing dispensing mechanism parameters with acceptable dispending mechanism parameters.

A sensor can be configured to measure the at least one administration parameter. Such a sensor is also referred to herein as an “administration sensor.” One or more administration sensors can be used. This measurement generates administration data which the drug administration system or drug administration device can compare with the acceptable administration parameters. The at least one administration sensor can be configured to measure the at least one administration parameter simultaneously with the at least one dispensing sensor measuring the at least one dispensing mechanism parameter. The at least one administration sensor can be configured to measure the at least one administration parameter after the at least one dispensing sensor measures the at least one dispensing mechanism parameter. Measuring the at least one administration parameter may allow for confirmation that the drug has been successfully administered and may act as an independent check on the confirmation that the operation of the dispensing mechanism is complete. Measuring the at least one administration parameter may also increase the chance of detecting unsuccessful administration, than is the at last one administration parameter was not measured, and allow quicker intervention to prevent the patient being harmed.

A processor can be configured to receive the dispensing mechanism data and to determine whether the operation of the dispensing mechanism is complete based on the dispensing mechanism data. This determination may enable confirmation that the dispensing mechanism has operated as intended. The processor may be present as part of the drug administration device or as part of an external device that is external to the drug administration device and that can be located remote to the drug administration device.

As discussed herein the external device can be a device, which is distinct from the drug administration device, that comprises components required for determining the completion of the operation of the dispensing mechanism and comparing the at least one administration parameter with the acceptable administration parameters. Therefore, the external device can include a computer system that includes a memory for storing the acceptable administration parameters and a communications interface for receiving the data from the drug administration device. Accordingly, the drug administration device can include a corresponding communications interface configured to electronically send data, e.g., the drug administration data and/or the administration data.

In an exemplary embodiment, the external device can be a smart device, such as a smart phone, tablet, smart watch, etc., that can communicate wirelessly with the drug administration device.

Where the administration sensor is not part of the drug administration device, the administration sensor can include a communications interface for sending the administration data to either the drug administration device or an external device.

A second processor can be configured to receive the administration data from the at least one administration sensor and confirm whether the administration was successful when the operation of the dispensing mechanism is determined to be complete by the first processor. This confirmation enables the system or device to confirm that the drug has been administered successfully which may result in improved patient safety, as described in more detail above. The second processor may thus provide a safety feature by confirming whether delivery was successful or not. The second processor can be present on the drug administration device or an external device. In the absence of a second processor, the first processor can be configured to perform operations relating to both the at least one dispensing mechanism parameter and the at least one administration parameter. In particular, the first processor can be configured to perform all required processing functions.

In embodiments in which the second processor is present on an external device, in response to the administration being successful, the external device, e.g., the second processor thereof, can be configured to automatically trigger mailing (or other delivery as appropriate) of a new drug administration device to the patient (or to another site for patient pickup or use as appropriate) so that the patient can timely receive the new drug administration device before the next scheduled drug dose is due and/or so that the patient has a limited supply of the drug on hand at any given time. The patient having a limited supply of the drug on hand at any given time may be particularly important for controlled substances that could be abused and/or be more likely than other drugs to develop into an addiction. Some drug administration devices are one-time use devices, which can make automatically triggering mailing or other delivery of a new one-time use drug administration device particularly useful.

In embodiments in which the second processor is present on an external device, in response to the administration being successful, the external device, e.g., the second processor thereof, can be configured to automatically trigger scheduling of a pickup of the used drug administration device by an authorized agent. Some drug administration devices may be required or advisable to be picked up by an authorized agent after use for recycling and/or to help ensure that any drug remaining in the drug administration devices (whether due to non-use of a drug administration device or residual drug being left in a drug administration device after proper use thereof) is disposed of safely and is not accessed by any unauthorized persons, which may be particularly important for controlled substances such as esketamine and ketamine. The drug administration device can include a location sensor configured to sense geographic location via GPS or otherwise, and the drug administration device can be configured to transmit location data gathered by the location sensor to the second processor. The second processor can thus be able to know from where the used drug administration device should be picked up. It may be more efficient for the authorized agent to pick up multiple drug administration devices at once than to pick the drug administration devices up one at a time as the devices are used. The second processor may thus be configured to use sensed location data received from each of the drug administration devices to know when a minimum number of drug administration devices are ready for pickup at a particular site and only then, when the minimum number of drug administration devices are ready for pickup at a particular site, automatically trigger scheduling of a pickup of the used drug administration devices by an authorized agent.

The second processor can be configured to modify further operation of the drug administration device depending on the dispensing mechanism data and/or the administration data. As discussed above, enabling modification of further operation of the drug administration device allows adjustments to be made that may make it more likely for further administrations to be successful and may reduce further wastage of the administered drug.

The second processor can be configured to record real-time data during operation of the dispensing mechanism. The second processor can be configured to use the real-time data to determine if there are one or more safety concerns during the operation of the dispensing mechanism. The second processor can be configured to notify the user of any determined safety concerns. The one or more safety concerns can include too high back-pressure. The one or more safety concerns can include the flow rate being too fast. The one or more safety concerns may include the flow rate being too slow.

The second processor can be configured to, in response to the administration being successful to a patient, automatically trigger gathering data regarding one or more physiological parameters of the patient using one or more sensors. Examples of physiological parameters include blood sugar level (e.g., measurable using a glucose monitor, etc.), blood pressure (e.g., measurable using a blood pressure monitor, etc.), perspiration level (e.g., measurable using a fluid sensor, etc.), heart rate (e.g., measurable using a heart rate monitor, etc.), respiratory rate (e.g., measurable using a respiratory monitor, a heat sensor configured to be located near a nose or mouth and to use heat detection on the out-breath or detect in/out airflow movement, a pressure sensor configured to be located near a nose or mouth and to use pressure detection on the out-breath or detect in/out airflow movement, a spirometer, etc.), temperature, (e.g., using a temperature sensor, etc.), blood oxygenation level (e.g., using a blood oxygen sensor, etc.), sedation, disassociation, etc. Measuring the at least one physiological parameter of a patient may enable confirmation that the drug has been successfully administered to the patient, as the drug is having a physiological effect on the patient, and/or may facilitate monitoring of the patient's condition following drug administration. Some drugs require that a patient who has the drug administered thereto be monitored for a period of time following drug administration. The drug's Risk Evaluation and Mitigation Strategies (REMs), e.g., a REMS for esketamine, ketamine, or other controlled substance, can require this monitoring. Automatically triggering the measuring of the at least one physiological parameter of the patient after drug administration may help facilitate the required monitoring.

In some embodiments, the one or more sensors (e.g., patient sensors) configured to gather data regarding one or more physiological parameters of the patient may already be scheduled to gather the data following drug administration, e.g., as part of the patient's regular treatment and monitoring. In such instances, the second processor automatically triggering the one or more sensors configured to gather data regarding one or more physiological parameters of the patient can include causing the one or more sensors to gather data at particular elapsed time(s) after the drug administration and/or at a different regularly scheduled frequency (e.g., an increased regularly scheduled frequency, which may include continuous gathering of data) than previously scheduled for the one or more sensors. Gathering the data at particular elapsed time(s) after the drug administration (e.g., every ten minutes after drug administration, every twenty minutes after drug administration, every thirty minutes after drug administration, every hour after drug administration, once forty minutes after drug administration and again two hours after drug administration, once thirty minutes after drug administration and then every hour after drug administration, etc.) may help ensure that useful physiological parameter data is gathered for analysis of the drug's effect on the patient. Similarly, gathering the data at a different regularly scheduled frequency may help ensure that useful physiological parameter data is gathered for analysis of the drug's effect on the patient.

The second processor can be configured to modify the further operation of the drug administration device in any of the ways described herein. A notification output can be provided, as discussed above, to inform a user of the drug administration device that the further operation of the drug administration device has been modified. For example of further operation being modified, the second processor can be configured to prevent further operation of the drug administration device when the successful administration was not confirmed. This prevention of further operation prevents further unsuccessful administrations and means that the problem that caused the administration to be unsuccessful must be addressed before the drug administration device can again deliver a drug dose. The second processor being configured to prevent the further operation of the drug administration device can include the second processor being configured to disable a power supply of a motor of the drug administration device, such as by opening or closing a switch as discussed above; by the second processor being configured to cause enabling of a device operation prevention mechanism; and/or by the second processor being configured to cause a change of at least one variable parameter of an algorithm used in controlling drug administration from the drug administration device.

The at least one dispensing sensor or the at least one administration sensor can include any of a variety of sensors, such as a Hall effect sensor, a motion sensor, a pressure sensor, etc. As mentioned above, Hall effect sensor provides a reliable measurement of displacement since the Hall effect sensor being physically obscured by dust particles, or other means, will not affect the measurements.

For another example, the at least one dispensing sensor or the at least one administration sensor can include a volumetric flow meter. This enables a total volume of drug administered to be calculated and can therefore be used to confirm operation of the device or administration of the drug. The volumetric flow meter can include a piston meter. The volumetric flow meter can include an oval gear meter. The volumetric flow meter may be positioned in the vicinity of the outlet, or at the outlet, of the drug administration device.

For yet another example, the at least one dispensing sensor or the at least one administration sensor can include a pressure-based meter. The pressure-based meter can include a Venturi meter.

For another example, the at least one administration sensor can include a liquid detection sensor configured to measure an amount of liquid present in the vicinity of an injection site, as discussed herein.

For yet another example, the at least one administration sensor can be configured to monitor an angular orientation of the drug administration device, e.g., using an accelerometer, a gyro, a tilt/angle switch (mercury free), a position sensor, etc. As mentioned above, some drug administration devices should be at a particular angular orientation relative to the patient during drug administration to help ensure that the drug is delivered properly.

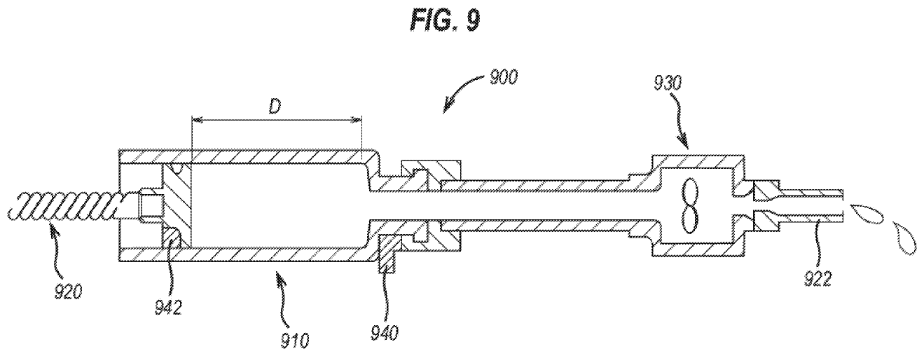

FIG. 9 is a schematic view of an embodiment of a drug administration device 900 which comprises a volumetric flow meter 930 and a Hall effect sensor 940. In this example, the drug administration device 900 is an implementation of the universal drug administration device 500 described herein. Any compatible drug administration device can be used in this example.

The drug administration device 900 comprises a drug holder 910 which retains a drug to be dispensed, and a dispensing mechanism 920 which is configured to dispense a drug from the drug holder 910 so that it can be administered to a user. In this example, the dispensing mechanism 920 is a plunger. The drug administration device 900 comprises a Hall effect sensor 940, and the dispensing mechanism 920 comprises a magnet 942. As displacement D of the dispensing mechanism 920 changes, the reading on Hall effect sensor 940 will change due to the change in proximity of magnet 942. The Hall effect sensor 940 may be calibrated such that each reading corresponds to a different displacement D. The readings of the Hall effect sensor 940 can therefore be used to confirm operation of the dispensing mechanism 920 or administration of the drug by confirming that the dispensing mechanism has moved an intended distance. In this example, the Hall effect sensor 940 configured to measure at least one dispensing mechanism parameter and output dispensing mechanism data relating to the at least one dispensing mechanism parameter. It will be understood by one skilled in the art that the position of the Hall effect sensor 940 is shown merely by way of an example, the Hall effect sensor 940 may be positioned anywhere that the readings of the Hall effect sensor 940 will change as the displacement D of the dispensing mechanism 920 changes. In an alternate configuration, the displacement mechanism 920 can include the Hall effect sensor 940, and the drug administration device 900 can include the magnet 942.

As a drug is dispensed by the dispensing mechanism 920 through a discharge nozzle 922, the drug passes through the volumetric flow meter 930. The volumetric flow meter 930 is configured to measure the amount of drug dispensed by the dispensing mechanism 920. In this example, the volumetric flow meter is one of the at least one administration sensors. It will be understood by one skilled in the art that the position of the volumetric flow meter 930 is shown merely by way of an example, the volumetric flow meter 930 may be positioned anywhere the drug passes through when the drug is being administered. The volumetric flow meter 930 may be positioned in a vicinity of an outlet of the drug administration device 900. By measuring the amount of liquid passing through the volumetric flow meter 930, it can be confirmed that administration of the drug has been successful.

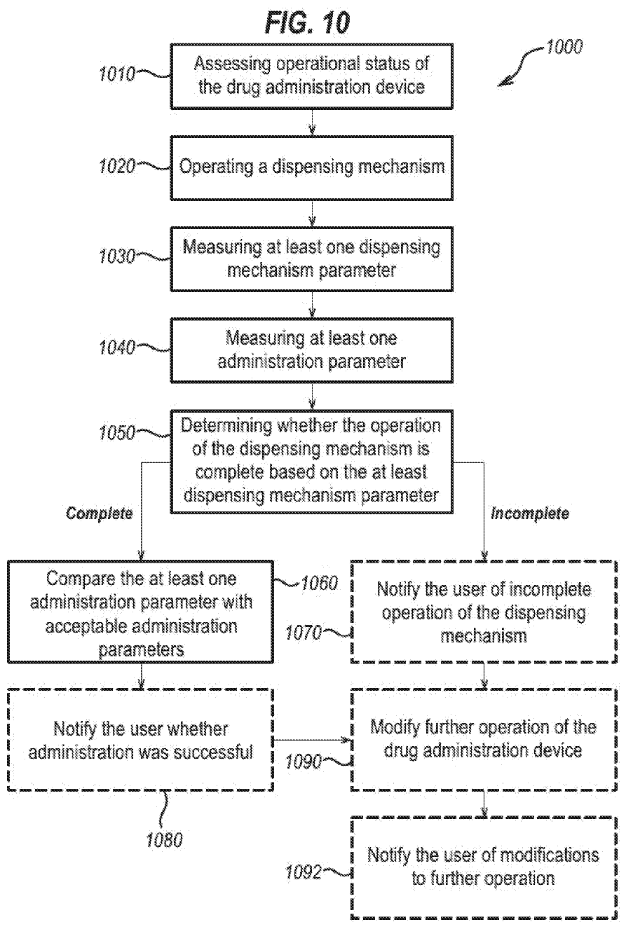

FIG. 10 illustrates a flow diagram showing an embodiment of a method 1000 of confirming administration from a drug administration device.

Optionally, an operational status of the drug administration device is assessed 1010 before operation of the device. A dispensing mechanism of the drug administration device then operates 1020. At least one dispensing mechanism parameter is measured 1030. The at least one dispensing mechanism parameter can be any dispensing mechanism parameter described herein. At least one administration parameter is measured 1040. The at least one administration parameter can be any administration parameter described herein. It is then determined whether the operation of the dispensing mechanism is complete based on the at least one dispensing mechanism parameter 1050.

When the operation of the dispensing mechanism is determined to be complete, the at least one administration parameter is compared with acceptable administration parameters in order to confirm whether administration was successful 1060. Then, optionally, the drug administration device's user can be notified whether administration was successful 1080. If the operation of the dispensing mechanism is determined to be incomplete then, optionally, the user is notified of the incomplete operation of the dispensing mechanism 1070. Optionally, after notifying the user whether administration was successful 1080, or notifying the user of the incomplete operation of the dispensing mechanism 1070, the further operation of the drug administration device can then be modified 1090. Optionally, the user may then be informed of modifications to further operation of the drug administration device 1092.

### Drug Delivery Conformance, Notification, And Prioritization

As outlined above, it can be desirable to monitor the delivery of drugs to identify delivery problems or other issues, particularly in relation to drug trials or conformity with dosing prescriptions. In an exemplary embodiment, a drug administration and monitoring system includes a drug administration device, a monitoring device, and a sensor. The drug administration device, the monitoring device, and the sensor can all be integrated with each other into a single device. Alternatively, the drug administration device and the monitoring device can both be integrated with each other into a single device, and the sensor can be a standalone device. In another alternative, the drug administration device, the monitoring device, and the sensor can each be standalone discrete devices.

The sensor can be configured for in vivo monitoring of the patient in real time and to sense at least one patient parameter. The sensor of the drug administration and monitoring system is thus also referred to herein as a “patient sensor.” The patient sensor can be configured to be placed on, in or against the patient, or in a vicinity of the patient. For example, the patient sensor can be integrated into a wearable device, such as a smart watch, etc., or be carried by the patient, for example by being integrated into a mobile user device, such as a smartphone, etc.

The monitoring device is formed as an electronic device, such as a computer system as described herein. In an exemplary embodiment, the monitoring device is a mobile computer system, e.g., mobile phone, smart watch, etc., which may allow for user access to information via the monitoring device at many different locations of the user.

The drug administration device, the monitoring device, and the patient sensor are each in data communication with each other. The communication can be one way (unidirectional), e.g., from the drug administration device to the monitoring device and from the patient sensor to the monitoring device. Alternatively, the data communication can be bidirectional.

The monitoring device can be configured to receive data pertaining to drug delivery events from the drug administration system, and to receive the at least one patient parameter from the patient sensor.

The monitoring device can be configured to log a drug delivery event, determine a drug response associated with the drug delivery event on a specific patient based on the at least one patient parameter which is sensed by the patient sensor, and determine and store data pertaining to a patient outcome associated with the drug response and the drug delivery event. The drug delivery event can be logged and/or the determined data can be stored in the patient's electronic health record (EHR) and/or in a form required for use with the drug that was administered such as a patient monitoring form for a particular drug's Risk Evaluation and Mitigation Strategies (REMS). Esketamine, ketamine, and other controlled substances typically have a REMS. The EHR and/or the form may therefore be accurately and timely updated.

The determined patient outcome can be one or more of a time period after administration of drug delivery for which the drug response is sensed, an intensity of the determined drug response at a given time or over a given time period after drug administration to the patient, a time duration for which the drug response in relation to the drug delivery event is determined, and effectiveness or response of the drug on the patient following the drug delivery event in relation to particular symptoms associated with a medical indication being treated by the drug.

The monitoring device can be configured to generate a notification to the patient and/or a remote patient monitoring device based on the determined patient outcome. The remote patient monitoring device can be an external device, as described herein.

The at least one patient parameter being sensed by the sensor can include one or more of temperature, pH level, a biomarker, glutathione level, skin thickness, subcutaneous tissue thickness, blood oxygen level, blood glucose level, blood pressure, heart rate, respiratory rate, sleep, and metabolic rate.

The monitoring device can be configured to check a conformity of the drug delivery event, and optionally one or more additional drug delivery events by the drug administration device, with a prescribed drug dosing scheme. The monitoring device can be further configured to generate a notification to the patient and/or the remote patient monitoring device if the drug delivery event and the optional one or more additional drug delivery events does not conform to the prescribed drug dosing scheme. The prescribed drug dosing scheme can specify one or more of the following drug dosing parameters: drug delivery rate, drug delivery duration, drug delivery volume, and drug delivery frequency.

The drug administration system can also include an environmental sensor configured to detect an external stimulus. The environmental sensor of the drug administration system can be configured to detect one or more of a user input to the drug administration device, geographical location, ambient temperature, pressure, and ultraviolet radiation level. The drug administration system can also include a user interface, the external stimulus can be the user input, and the user input can be input via the user interface.

The monitoring device can also be configured to determine, based on the sensed at least one patient parameter and/or the external stimulus, whether a likelihood of side effects associated with the drug has increased, and, if it is determined that the likelihood of side effects has increased, generate a notification to the patient and/or the remote patient monitoring device if the drug delivery event and the optional one or more additional drug delivery events does not conform to the prescribed drug dosing scheme. The monitoring device can include a device indicator, and the drug administration device can be configured to activate the device indicator if it is determined that the likelihood of side effects is increased.

The monitoring device can be configured to provide a plurality of notifications to the patient and/or the remote monitoring device pertaining to the drug delivery event, the optional one or more additional drug delivery events, and/or the at least one patient parameter, and the plurality of notifications can be notified in order according to a predefined priority order based on the detected drug delivery event and optional one or more additional drug delivery events and/or based on the at least one patient parameter.

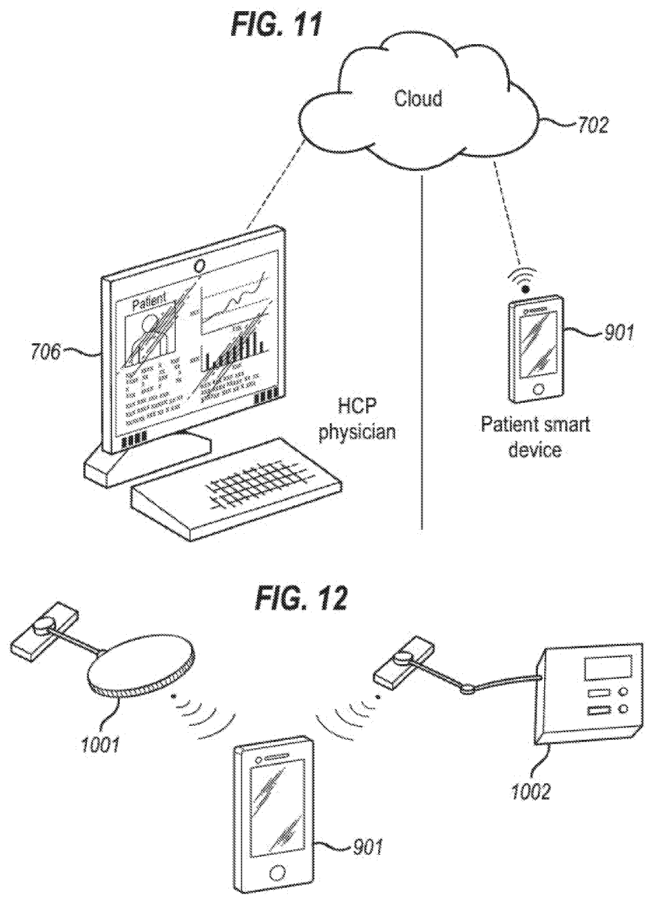

FIG. 11 depicts an embodiment of a drug administration and monitoring system including a monitoring device 901 which is a smart monitoring device for the patient and that is in communication with the drug administration device 500 or housing 630 (FIGS. 5-7), and with the central system 700 (FIG. 7). The monitoring device 901 is configured to monitor drug delivery events of the drug administration device 500 or housing 630, and is configured to send data pertaining to the drug delivery events to the central system 700.

As shown in FIG. 12, the drug administration and monitoring system also includes one or more patient sensors 1001 in communication with the monitoring device 901 and one or more environmental sensors 1002 in communication with monitoring device 901. As mentioned above, the patient sensor(s) 1001 can be configured to sense one or more current conditions of a patient including any one or more of temperature, pH level, a biomarker, glutathione level, skin thickness, subcutaneous tissue thickness, blood oxygen level, blood glucose level, blood pressure, heart rate, respiratory rate, sleep, and metabolic rate. As also mentioned above, the environmental sensor(s) 1002 (e.g., one or more of the environment sensor 94, the location sensor 98, and the device sensor 92 of FIG. 5B or FIG. 6) can be configured to sense one or more of a user input to the drug administration device, geographical location, ambient temperature, pressure, and ultraviolet radiation level.

One embodiment of a patient sensor is depicted in FIG. 14. The drug administration device 500 (FIG. 5B), which in this exemplary embodiment of FIG. 14 is an autoinjector, includes a light source 1201 located at a distal end 1200 of the device 500. The patient sensor is a light detector 1202 configured to detect reflected light back from skin 1203 of the patient when the light source 1201 is activated to transmit light 1201a onto the skin 1203 of the patient. Depending on characteristics of the light received back (e.g., intensity, variation, etc.) the one or more patient parameters (e.g., skin thickness, heart rate, etc.) can be determined. A person skilled in the art will appreciate how reflected light can be analyzed, e.g., by a processor of the monitoring device 901 or other processor, to determine the one or more patient parameters sensed via the reflected light.

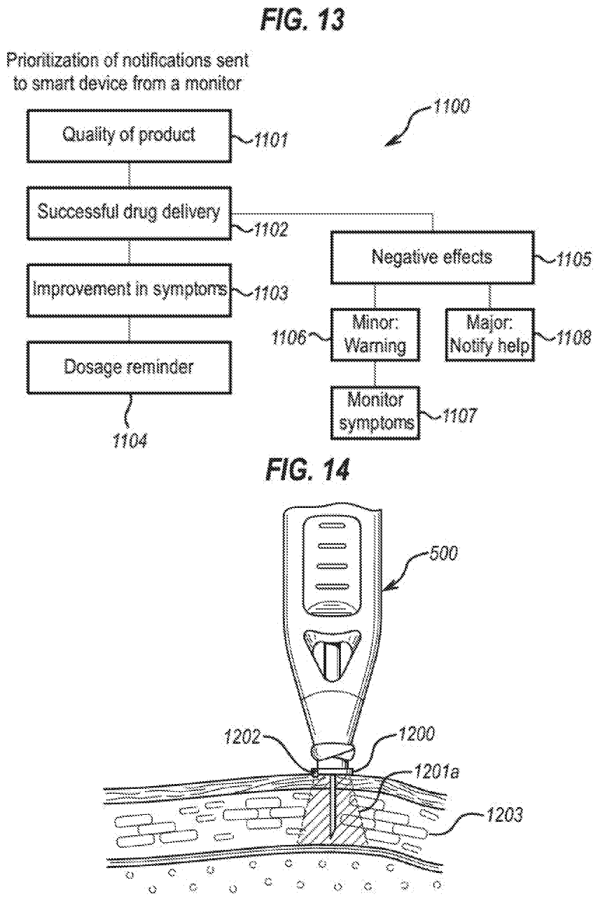

The drug administration device 500 and/or the housing 630 at the home base 708, the medical facility 706, and/or the mobile location 710 (FIG. 7) can be configured to receive notifications from the monitoring device 901 and/or the central system 700 (FIG. 7) in relation to the drug delivery events. In particular, the notifications can pertain to any one or more of quality of drug product delivered, successful drug delivery, unsuccessful drug delivery, whether or not self-calibration of the drug administration device 500 occurred between drug deliveries, time and duration of drug delivery events, conformity with prescribed drug delivery profiles, unusual or non-prescribed drug delivery for the particular drug, detected symptoms of a medical indication being treated by the drug, detected side effects, environmental parameters associated with drug delivery, patient parameters, emergency events (such as over or under dosing of drug delivery), and errors with drug delivery. Such emergency events of drug delivery detected by the monitoring device 901 can be alerted to, e.g., the patient and/or the patient's care giver. The notifications can be prioritized and notified in a predetermined order according to a predetermined priority matrix 1100, for example a priority order based on risk to the patient and/or conformance with a drug trial, as shown in an embodiment in FIG. 13. Notifications can be alerted by sound or visual alerts on the monitoring device 901 itself, and/or on the drug administration device 500 and/or the housing 630.

In the exemplary notification sequence 1100 of FIG. 13, at step 1101 a user is notified of a quality of the drug (referenced in FIG. 13 as the drug product). At step 1102 the user is informed of successful drug delivery. If an improvement in symptoms of the patient (which may be the user) is recorded then the user is notified at step 1103, after which the user is reminded to take a further dosage at step 1104. If, after the successful drug delivery at step 1102, negative effects associated with the drug delivery are detected then the user is notified at step 1105. If it is determined that the negative effect is minor, then the user is warned at step 1106, and symptoms are further monitored at step 1107. Alternatively, if the determined negative effects are major, then at step 1108 the user is notified to seek help.

Referring again to FIG. 11, in some embodiments the monitoring device 901 is configured to monitor drug delivery events of a drug holder and thereby monitor drug delivery of the drug administration device 500 or housing 630 that is used with the drug holder. Knowing when the drug holder is first used may be useful in evaluating patient compliance, in ensuring that drugs are used before their expiration date (e.g., by a processor comparing a date/time of first use with a known expiration date of the drug), and/or in determining whether drug administration occurred successfully. The drug holder is configured to send data pertaining to the drug delivery events to the central system 700. Thus, in such embodiments, the drug administration device 500 or housing 630 can, but need not be, configured to send data pertaining to the drug delivery events to the central system 700.

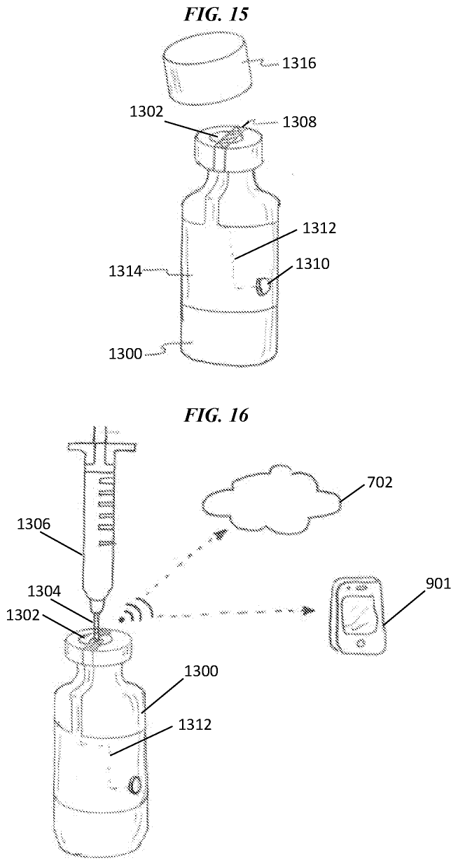

FIGS. 15 and 16 depict an embodiment of a drug holder 1300 that can be included in the drug administration and monitoring system. The drug holder 1300 in this illustrated embodiment is a vial configured to hold a drug in a liquid dosage form therein. The drug is obscured in FIGS. 15 and 16. The drug holder 1300 includes a septum 1302 configured to be punctured to allow access through the septum 1302 to the drug in the drug holder 1300. FIG. 16 depicts an embodiment of a needle 1304 of a syringe 1306 configured to be inserted through the septum 1302. FIG. 16 shows the needle 1304 extending through the septum 1302 and into the drug holder 1300. After drug is drawn into the syringe 1306 from the drug holder 1300 through the needle 1304, the needle 1304 can be withdrawn from the septum 1302 to allow for drug delivery to a patient using the syringe 1302.

The drug holder 1300 also includes a circuit trace 1308 and a chip 1310 in electronic communication 1312 with the circuit trace 1308. The circuit trace 1308 and the chip 1310 are integrated with or otherwise attached to a label 1314 configured to be adhered or otherwise applied to an external surface of the drug holder 1300. The label 1314 can have any of a variety of sizes and shapes. Also, the circuit trace 1308 and the chip 1310 can be attached to the drug holder 1300 in other ways. The chip 1310 is generally configured as a computer system and includes a power source and a communication interface. The circuit trace 1308 is positioned over the septum 1302 and is configured to be punctured by a needle inserted through the septum 1302. FIG. 15 shows the circuit trace 1308 in an unbroken state before being punctured. With the circuit trace 1308 in the unbroken state, the electronic communication 1312 between the circuit trace 1308 and the chip 1310 is unbroken. FIG. 16 shows the circuit trace in a broken state after being punctured, with the puncturing in this illustrated embodiment being by the needle 1304 of the syringe 1306. With the circuit trace 1308 in the broken state, the electronic communication 1312 between the circuit trace 1308 and the chip 1310 is broken. In other words, the circuit trace 1308 being in the broken states “breaks” electronic communication 1312 between electronic components. In response to the circuit trace 1308 moving from the unbroken state to the broken state, e.g., in response to the electronic communication 1312 being “lost,” the communications interface of the chip 1310 is configured to communicate drug delivery event data to the cloud 702 and/or the monitoring device 901. This drug delivery event data indicates that the drug holder 1300 has been used for the first time and that the drug administration process is thus likely commencing, as reflected by the septum 1302 having been pierced and the circuit trace 1308 having been “broken” by the puncturing. The circuit trace 1308 remains in the broken state after the needle or other member that punctured the septum 1302 is withdrawn from the septum 1302. The circuit trace 1308 moving from the unbroken state to the broken state is thus indicative of a first use of the drug holder 1300.

The drug holder 1300 can include a cap 1316 configured to be positioned over the septum 1302 and to be removed from the drug holder 1300 by a user prior to insertion of a needle or other member through the septum 1302. The cap 1316 may thus provide protection to the circuit trace 1308 and to the septum 1302 to help prevent the circuit trace 1308 from prematurely moving from the unbroken state to the broken state, e.g., during shipping or handling. The cap 1316 can be removably attached to the drug holder 1300 in any number of ways, as will be appreciated by a person skilled in the art, such as via threading, a hinge configured to allow the cap 1316 to be flipped off, a snap fit, etc.

Referring again to FIG. 11, in some embodiments the monitoring device 901 is configured to monitor drug mixing as a drug delivery event. Knowing information related to drug mixing, e.g., when or whether drug mixing begins and when or whether drug mixing ends, etc., may be useful in determining whether drug administration occurred successfully, such as by checking whether mixing occurred for a predetermined minimum amount of time known for drugs to be properly mixed together, etc.

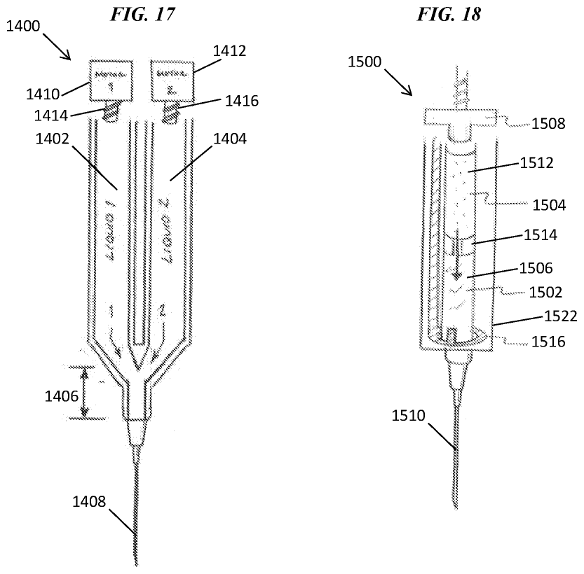

FIG. 17 depicts an embodiment of a drug administration device 1400 as the drug administration device 500 that can be included in the drug administration and monitoring system. The drug administration device 1400 is configured to mix a first drug 1402 and a second drug 1404 on board the device 1400. The first and second drugs 1402, 1404 are each a liquid in this illustrated embodiment. The first and second drugs 1402, 1404 are different from one another and are mixed to form a drug in a mix chamber 1406 of the drug administration device 1400. The mixed drug is deliverable from the mix chamber 1406 of the drug administration device 1400.

The drug administration device 1400 in this illustrated embodiment is a syringe that includes a plunger configured to drive the mixed drug from the mix chamber 1406 and out a needle 1408 of the drug administration device 1400. The drug administration device 1400 can have other components as discussed herein for syringes and for the drug administration device 500.

FIG. 17 shows the drug administration device 1400 as including a first motor 1410 and a second motor 1412. The first motor 1410 is configured to drive the first drug 1402 into the mix chamber 1406, e.g., by driving a first plunger 1414 (partially shown) of the device 1400 distally, which is downward in the view shown in FIG. 17. The second motor 1412 is configured to drive the second drug 1404 into the mix chamber 1406, e.g., by driving a second plunger 1416 (partially shown) of the device 1400 distally. The drug administration device 1400 can include a processor configured to control the motors 1410, 1412 and thus control the mixing of the drugs 1402, 1404. The motors 1410, 1412 can be configured to drive equal amounts of the first and second drugs 1402, 1404 into the mix chamber 1406 and to drive the first and second drugs 1402, 1404 into the mix chamber 1406 at a same rate as one another. Alternatively, the motors 1410, 1412 can be configured to drive different amounts of the first and second drugs 1402, 1404 into the mix chamber 1406 and/or to drive the first and second drugs 1402, 1404 into the mix chamber 1406 at a different rates from one another. Depending on one or more factors such as the desired concentration of the mixed drug, the types of the first and second drugs 1402, 1404, etc., driving different amounts the first and second drugs 1402, 1404 into the mix chamber 1406 and/or driving amounts (same or different) of the first and second drugs 1402, 1404 into the mix chamber 1406 at different rates may result in the most easily injected, most evenly combined, etc. mixed drug in the mix chamber 1406.

One or more types of data pertaining to the drug delivery events as related to the mixing of the first and second drugs 1402, 1404 can be communicated from the drug administration device 1400 to the cloud 702 and/or the monitoring device 901. Examples of the data include a start date/time of the first motor 1402, a stop date/time of the first motor 1402, a speed of the first motor 1402 during driving of the first plunger 1414, a current of the first motor 1402 during driving of the first plunger 1414, a start date/time of the second motor 1404, a stop date/time of the second motor 1404, a speed of the second motor 1404 during driving of the second plunger 1416, and a current of the second motor 1404 during driving of the second plunger 1416.

FIG. 18 depicts an embodiment of a drug administration device 1500 as the drug administration device 500 that can be included in the drug administration and monitoring system. The drug administration device 1500 is configured to mix a first drug 1502 and a second drug 1504 on board the device 1500. In this illustrated embodiment, the first drug 1502 is a liquid, and the second drug 1504 is a solid, e.g., a powder or other solid. The first and second drugs 1502, 1504 are different from one another and are mixed to form a drug that is deliverable from the drug administration device 1500. In this illustrated embodiment, a chamber 1506 in which the first drug 1502 is disposed prior to mixing serves as the mix chamber where the first and second drugs 1502, 1504 are mixed together.

The drug administration device 1500 in this illustrated embodiment is a syringe that includes a plunger 1508 configured to drive the second drug 1504 from its initial chamber 1512 and into the mix chamber 1506. The drug administration device 1500 includes a motor configured to drive the plunger 1508. The plunger 1508 is configured to break a seal 1514 as the plunger 1508 moves distally (downward in the view of FIG. 18) in driving the second drug 1504. The seal 1514 initially separates the chambers 1506, 1512 and keeps the drugs 1502, 1504 separate from one another prior to a time of desired mixing. The drug administration device 1500 also includes a needle 1510 through which the mixed drug can exit the drug administration device 1500. The drug administration device 1500 can have other components as discussed herein for syringes and for the drug administration device 500.

FIG. 18 shows the drug administration device 1500 as including an agitator 1516 configured to be driven by a motor of the drug administration device 1500 (which can be the same motor that drives the plunger 1508 or a different motor). The agitator 1516 is configured to move relative to a housing 1522 of the drug administration device 1500 to cause movement, e.g., vertical movement, horizontal movement, rotational movement, or some combination thereof) of the mix chamber 1506 (and the other chamber 1512). The movement of the mix chamber 1506 causes the first and second drugs 1502, 1504 in the mix chamber 1506 to mix together. The plunger 1508 that has moved distally to drive the second drug from the chamber 1512 into the mix chamber 1506 can serve as a proximal end of the mix chamber 1506 during mixing (e.g., during movement of the agitator 1516). Depending on one or more factors such as the desired concentration of the mixed drug, the types of the first and second drugs 1502, 1504, etc., the agitator 1516 can move at different speeds and/or for different lengths of time to result in the most easily injected, most evenly combined, etc. mixed drug in the mix chamber 1506.

One or more types of data pertaining to the drug delivery events as related to the mixing of the first and second drugs 1502, 1504 can be communicated from the drug administration device 1500 to the cloud 702 and/or the monitoring device 901. Examples of the data include a start date/time of plunger 1508 movement to drive the second drug 1504, a stop date/time of plunger 1508 movement to drive the second drug 1504, a start date/time of plunger 1508 movement to drive the mixed drug from the mix chamber 1506 and out the needle 1510, a stop date/time of plunger 1508 movement to drive the mixed drug from the mix chamber 1506 and out the needle 1510, a speed of the motor during driving of the plunger 1508 and/or the agitator 1516, and a current of the motor during driving of the plunger 1508 and/or the agitator 1516.

### Stimuli Responsive Drug Administration Device for Local Drug Activation

In another exemplary embodiment, a drug administration system includes a drug administration device that includes a drug holder configured to hold a drug. The drug administration device also includes a dispensing mechanism configured to dispense the drug. The drug administration system also includes a first sensor configured to sense a patient parameter. The drug administration system is configured to locally activate the drug at a target location in the patient after the drug has been dispensed by the dispensing mechanism and administered to the patient. The local activation is responsive to the patient parameter and an external stimulus.

Local activation may allow an inactive form of a drug, or a drug with attenuated activity, to be administered systemically to a patient. The drug is configured to only be made active at the target location, where its therapeutic effect is desired, in the patient. The location in the patient is to be understood to include locations on a surface of the patient, such as the skin. Advantageously, harmful effects that may be associated with the active form of the drug on unintended, or off-target, locations in the patient are minimized. In addition to the aforementioned safety benefits, efficacy of the drug may also be improved, as the drug is only activated at the target location (which is typically a volume rather than a specific point) within the patient's body where and when the drug's therapeutic effect is desired, thus concentrating the drug's benefit. Consequently, the dose of the drug required may also be reduced. By making the local activation responsive to the sensed patient parameter and the external stimulus, efficacy and safety may be further improved, as the drug is only activated when the drug administration device determines that conditions, e.g., the patient parameter and the external stimulus, are suitable or appropriate, or that sufficient time has elapsed such that the drug will have localized at the desired target location within the patient. The local activation being responsive to the sensed patient parameter and the external stimulus may also improve compliance, as the drug administration device controls when the drug becomes active in accordance with suitable or appropriate conditions, rather than being entirely dependent on when the drug is administered by a user. This activation may be particularly important for applications outside of a clinical setting, in which the drug may be administered by the patient themselves, instead of by a medical professional, and drug administration may be done at a sub-optimal time and/or under sub-optimal conditions. Certain sub-optimal conditions (e.g., improper temperature of the drug, improper pH level of the drug, elevated glutathione level, too low blood pressure, etc.) may result in the drug, in its usual dosage, not being effective, and/or may lead to increased side effects. Thus, it is beneficial if local activation of the drug is responsive to these suitable or appropriate conditions.

The local activation being responsive to the patient parameter and the external stimulus can include that the activation occurs when the patient parameter and the external stimulus satisfy a predetermined criterion. The predetermined criterion can be that the patient parameter and the external stimulus exceed or fall below a threshold level, or alternatively that the patient parameter and the external stimulus satisfy a predetermined mathematical relationship. An extent of the local activation can also be responsive to the patient parameter and the external stimulus.

The first sensor includes a device configured to detect or measure a physical property, or parameter, associated with the patient. The first sensor can be integral to the drug administration device and can be disposed on a surface of the drug administration device. Alternatively, the first sensor can be freely movable independent of the rest of the drug administration device to allow more convenient measurement of the patient parameter. The first sensor is configured to communicate, via wires or wirelessly, with other component(s) of the drug administration device, thereby enabling the local activation to be responsive to the first sensor's output.

The drug administration device can also include a second sensor configured to sense the external stimulus. The second sensor can be integral to the drug administration device and can be disposed on a surface of the drug administration device. Alternatively, the second sensor can be freely movable independent of the rest of the drug administration device to allow more convenient measurement of the external stimulus, with the second sensor being in communication, via wires or wirelessly, with other component(s) of the drug administration device.

In general, the external stimulus is a physical property that is external to the patient. For example, the external stimulus can be an environmental parameter. An environmental parameter is a characteristic of a local environment of the patient. For example, the environmental parameter can be ambient temperature or ambient pressure.

The second sensor may permit identification and/or quantification of the external stimulus, such as an environmental parameter, that can influence a localization time, efficacy, and/or side effects associated with the drug. For example, ambient temperature may influence viscosity of the drug, which in turn influences a time required for the drug to reach, or localize, at the target location in the patient. The drug may become less viscous if t the drug to be too warm (e.g., if the drug's temperature is above a predetermined threshold temperature or is outside a predefined safe range of temperatures), and can in turn travel more freely in the patient, at greater speed, to the target location. Increased temperature of the drug can also lead to increased heart rate and vasodilation, thereby leading to faster localization of the drug at the target site. The drug's temperature settles to the environmental temperature, so the environmental temperature can be indicative of the drug's temperature. Consequently, the local activation being responsive to such an external stimulus may improve efficacy and/or minimize side effects.

The drug administration device can include an energy source configured to provide energy to locally activate the drug at the target location in the patient. This provision of energy can have the effect that the drug can be activated at a precise location by targeted application of energy to a patient. The energy source can be configured to target not only a surface location on the patient, but also be set to a desired penetration depth, to provide a precise target location, (which is typically a volume rather than a specific point), within the patient. The energy source can include multiple energy sources of different types to provide different penetration and activation characteristics.

The drug can be configured to interact with the energy provided by the energy source to assume its active form. Alternatively, an activation device implanted in the patient may trigger activation of the drug at the target location in response to energy provided by the energy source.

An amount of energy provided by the energy source can be responsive to the patient parameter and the external stimulus. This responsiveness of the amount of energy can have the effect that an extent, and thus a rate, of drug activation may be more precisely controlled to improve the drug's efficacy and safety in response to the patient parameter and the external stimulus. For example, it may be desirable to more gradually activate the drug by providing a smaller amount of energy over a longer period of time, if the drug administration device determines that conditions (as indicated by the patient parameter and the external stimulus) are such that the drug cannot be taken up (e.g., absorbed, metabolized, etc.) by the patient at the target location as quickly as normal. Thus, a further benefit of the gradual activation may be that less of the drug is wasted.

The energy source can include one or more of a light source, an ultra-sound source, an electro-magnetic field source, and a radioactive material.

The energy source can be configured to interact with the drug or an implanted device, as mentioned above. As mentioned above, a combination of multiple types of energy sources can be provided to provide variable penetration characteristics. For example, energy sources capable of providing electro-magnetic fields of different wavelengths can be used. Where appropriate, a frequency of the energy source can be adjusted by the drug administration device to control a rate and amount of energy delivery, as well as the penetration depth. Each energy source can be provided as a separate unit to the drug administration device or as an integral part of the drug administration device.

The drug administration device can be configured to administer a chemical activation agent to the target location in the patient to locally activate the drug. While this chemical activation agent administration requires the drug administration device to be capable of administering both a drug and the chemical activation agent, which may require an additional holder for the chemical activation agent and an additional associated dispensing mechanism, such a drug administration device advantageously does not require an energy source to activate the drug, and thus may be of simpler construction in certain respects. The additional holder for the chemical activation agent can be arranged in series with the drug holder, such that the same dispensing mechanism can be used for dispensing both the drug and the chemical activation agent. Alternatively, the holders can be arranged in parallel with independent dispensing mechanisms.

The chemical activation agent can be administered to the target location before or after the drug is administered to the patient by the drug administration device. For example, a chemical activation agent can be administered into a tumor before or after a chemotherapy drug, in an inactive form or with attenuated activity, is systemically delivered to the patient. The chemical activation agent can be configured to remain in the tumor so that the chemotherapy drug is only activated at the target location by a chemical reaction with, or a chemical reaction triggered by, the chemical activation agent.

The patient parameter sensed by the first sensor can include one or more of temperature, pH level, a biomarker, glutathione level, skin thickness, subcutaneous tissue thickness, blood oxygen level, blood glucose level, blood pressure, heart rate, and metabolic rate. For example, the patient parameter including pH level may be beneficial since certain drugs can be less effective if the pH level is above or below a certain level, and thus it may be beneficial to delay administering the drug until the pH level has returned to or is in a desired range, or to enhance activation to compensate for sub-optimal conditions. For another example, in the case of the patient parameter being a biomarker, the biomarker can be a naturally occurring molecule, gene, or other characteristic which provides an indicator as to a state of a particular pathological or physiological process or disease. Various sensors capable of sensing biomarkers, such as with microfluidics, and in various forms, such as skin patches, are known to a person skilled in the art. For yet another example, glutathione levels are of particular interest for chemotherapy drugs, as elevated levels of glutathione in cells can have the effect of protecting cells from the chemotherapy drugs. Thus, delaying activation until glutathione levels are reduced to below an acceptable threshold level, or enhancing the extent of activation to compensate for the increased protection of cells by glutathione, may be beneficial. For another example, skin thickness and subcutaneous thickness measurements can be used to ensure that activation, such as by an energy source, penetrates to sufficient depth. For still another example, various parameters relating to blood circulation, e.g., blood oxygen level, blood pressure, heart rate, and metabolic rate, influence the efficacy and safety of the drug, and thus adjustment of activation according to a value of one or more of the parameters relating to blood circulation may enhance efficacy and safety.

The external stimulus can include one or more of a user input, geographical location, ambient temperature, pressure, and ultraviolet radiation level. The local activation being responsive to the external stimulus may ensure that external factors, which may include a user's input, indicating the user's readiness to receive the drug, or other environmental parameters, are taken into consideration to optimize the timing or extent of local activation. Environmental parameters can impact on the efficacy or safety of the drug, and thus it may be advantageous to adjust activation accordingly. For example, certain drugs can cause elevated side effects at high temperatures, and thus it may be beneficial to delay or reduce the extent of activation in such circumstances.

The drug administration device can include a user interface, as discussed herein. The external stimulus can include a user input inputted via the user interface. As discussed above, the user interface can take the form of a touch screen and/or one or more buttons to allow the user to provide an input, such as to indicate the user's readiness to deliver and/or activate the drug as the external stimulus.

The drug administration device can be configured to administer the drug to the patient according to a drug dosing scheme. Such a drug dosing scheme can be pre-set by a doctor or other healthcare professional based on needs of the patient and can be based on parameters such as the patient's body weight, height, and age, to provide a starting drug dosing scheme which is likely to be effective for the patient. The drug dosing scheme can specify one or more of the following drug dosing parameters drug delivery rate, drug delivery duration, drug delivery volume, and drug delivery frequency.

The drug administration device can include an injector, as discussed above, and the drug dosing scheme can specify one or more of the following dosing parameters: a discharge nozzle advance depth of a discharge nozzle of the injector during administration of the drug to the patient, a discharge nozzle velocity of the discharge nozzle of the injector during administration of the drug to the patient, and the discharge nozzle acceleration of a discharge nozzle of the injector during administration of the drug to the patient. The discharge nozzle can be a needle of a syringe. Thus, the advance depth can be an exposure of the needle beyond a housing of the injector.

The drug dosing scheme can be based on the patient parameter and the external stimulus, which may allow the parameters of the drug dosing scheme to be further optimized according to factors which affect the efficacy and safety of the drug.

The patient parameter can include subcutaneous tissue thickness, as mentioned above, and the drug administration device can be configured to adjust a discharge nozzle advance depth based on the sensed subcutaneous tissue thickness when the drug administration device includes an advanceable discharge nozzle. This adjustment may ensure that the drug can be administered to the patient into tissue where the drug will be more readily absorbed, injection site leakage can be minimized, back flow of the drug can be prevented, and a risk of tissue damage and scarring resulting from the drug administration can be reduced.

The drug administration device can be configured to determine, based on the patient parameter and/or the external stimulus, whether a likelihood of side effects associated with the drug has increased, and, if it is determined that the likelihood of side effects has increased, adjust the drug dosing scheme to reduce the dosage of the drug to be administered and/or adjust activation means, which is configured to provide the local activation, to reduce local activation of the drug. This adjustment of the drug dosing scheme may minimize a risk of increased side effects under conditions which cause the side effects to be enhanced, such as high body or ambient temperature, by reducing the amount of drug that is administered, or by reducing the activation of the drug, since the active form of the drug may be associated with the side effects. The adjustment of activation may include stopping activation altogether, or reducing the extent to which activation occurs, such as by reducing energy provided by an energy source.

The drug administration device can include a device indicator, as discussed above, and the drug administration device can be configured to activate the device indicator if it is determined that the likelihood of side effects is increased. This notification may be used to alert the user to change their usage of the drug administration device, such as to cease operation of the drug administration device, or to alter a parameter of the drug administration device, such as the discharge nozzle advance depth when the drug administration device includes an advanceable discharge nozzle. As discussed above, the indicator may be an audible indicator, a visual indicator (such as an LED), or a tactile indicator (such as a lock-out mechanism or a vibration).

The drug administration device can be used as part of a drug administration system including the drug administration device and a drug capture and release mechanism configured to be implanted in a body of a patient. A variety of drug capture and release mechanisms, such as those used to capture a pill in the stomach, and release the pill into the digestive tract upon activation, are known to the skilled person in the art.

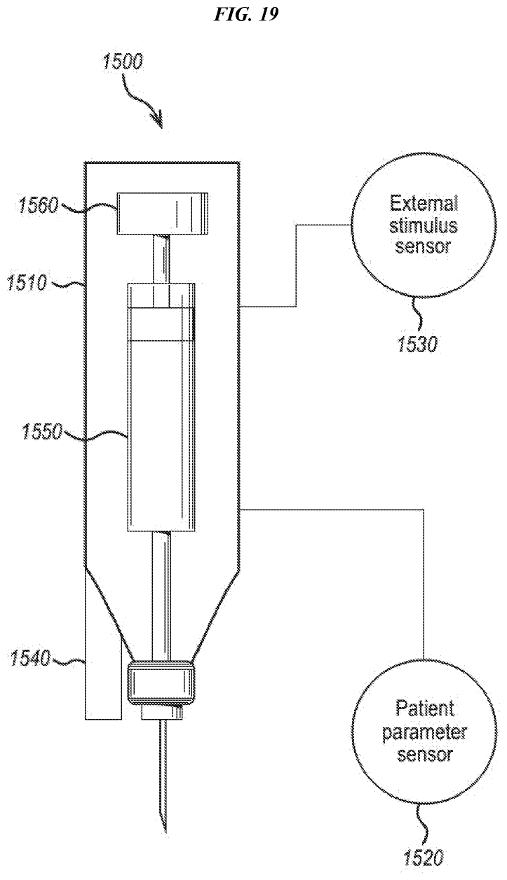

In an exemplary embodiment, as shown in FIG. 19, a drug administration system 1500 includes a drug administration device 1510, which in this illustrated embodiment is in the form of an autoinjector. The drug administration device's drug holder is in the form of a container 1550 which retains a drug to be dispensed, such as a syringe or vial. The drug administration device's dispensing mechanism includes a drive element 1560, which may include a piston and/or a rod, and a drive mechanism, as described above.

A patient sensor 1520 is discrete from the autoinjector 1510 and is connected, by a wire or wirelessly, to the autoinjector 1510, in order to communicate data. Alternatively, the patient sensor 1520 can be disposed on a surface of the autoinjector 1510 and arranged in close proximity to the patient's skin when the drug administration device 1510 is positioned for administration of the drug to the patient.

The skin is pricked by a user to release a small quantity of blood, and the patient sensor 1520 is configured to measure a blood glucose level in a sample of the blood disposed on the sensor 1520. An energy source 1540, in the form of an electro-magnetic field source in this illustrated embodiment, is arranged on a housing of the autoinjector 1510 to direct an electro-magnetic field towards a target location in the patient in order to activate the drug, which is insulin in this illustrated embodiment, after the drug has been administered, at the target location in the patient. An external stimulus sensor 1530 in this illustrated embodiment is in the form of a temperature sensor, is disposed at a position remote from the patient, and is configured to measure ambient temperature. A frequency of the electro-magnetic field delivered by the energy source 1540 is configured to be altered by the drug administration device 1510, e.g., to change the amount of energy delivered, in response to the measured blood glucose level and the measured ambient temperature. In particular, the measured values are compared with a look-up table to determine the frequency to be used. The frequency determines penetration of the energy and the extent of activation.

In an alternative embodiment, the autoinjector 1510 can be used to administer a chemotherapy drug, the patient sensor 1520 can be a blood pressure meter, and the external stimulus sensor 1530 can be used to measure ambient temperature. Data collected by the patient sensor 1520 and the external stimulus sensor 1530 can be transmitted to the autoinjector 1510 via wires or wirelessly. A processor on board the autoinjector 1510 can be used to calculate, based on the sensed blood pressure data and the measured ambient temperature, how long to delay initiating activation of the chemotherapy drug after the drug has been administered to the patient. Such a calculation may be based on an algorithm, or alternatively derived from a look-up table. The delay can be calculated such that the activation, provided by the energy source 1540 in the form of a light source 1540, directed at the tumor site coincides with the localization time for the drug to reach the target location in the patient. Since the drug is carried to the target site in the blood stream, the localization time is dependent on blood pressure, as well as ambient temperature, which affects various physiological parameters of the patient and characteristics of the drug itself, such as viscosity.

All of the devices and systems disclosed herein can be designed to be disposed of after a single use, or they can be designed to be used multiple times. In either case, however, the devices can be reconditioned for reuse after at least one use. Reconditioning can include any combination of the steps of disassembly of the devices, followed by cleaning or replacement of particular pieces, and subsequent reassembly. In particular, the devices can be disassembled, and any number of the particular pieces or parts of the device can be selectively replaced or removed in any combination. Upon cleaning and/or replacement of particular parts, the devices can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

It can be preferred that devices disclosed herein be sterilized before use. This can be done by any number of ways known to those skilled in the art including beta or gamma radiation, ethylene oxide, steam, and a liquid bath (e.g., cold soak). An exemplary embodiment of sterilizing a device including internal circuitry is described in more detail in U.S. Pat. Pub. No. 2009/0202387 published Aug. 13, 2009 and entitled “System And Method Of Sterilizing An Implantable Medical Device.” It is preferred that device, if implanted, is hermetically sealed. This can be done by any number of ways known to those skilled in the art.

The present disclosure has been described above by way of example only within the context of the overall disclosure provided herein. It will be appreciated that modifications within the spirit and scope of the claims may be made without departing from the overall scope of the present disclosure.

What is claimed is:

1. A method for confirming administration from a drug administration device, the method comprising:
operating a dispensing mechanism of the drug administration device;
measuring at least one dispensing mechanism parameter;
determining whether the operation of the dispensing mechanism is complete based on the at least one dispensing mechanism parameter;
measuring at least one administration parameter; and
when the operation of the dispensing mechanism is determined to be complete, comparing the at least one administration parameter with acceptable administration parameters in order to confirm whether the administration was successful.

2. The method of claim 1, further comprising:
modifying further operation of the drug administration device based on the at least one dispensing mechanism parameter and/or the at least one administration parameter.

3. The method of claim 2, further comprising:
notifying a user that the further operation of the drug administration device has been modified.

4. The method of claim 3, wherein notifying the user that the further operation of the drug administration device has been modified comprises one or more of a visual feedback, an auditory feedback, and a tactile feedback.

5. The method of any one of claims 2 to 4, wherein modifying the further operation of the drug administration device comprises:
preventing the further operation of the drug administration device when the successful administration was not confirmed.

6. The method of any one of claims 2 to 4, wherein modifying the further operation of the drug administration device comprises:
modifying a dosage volume to be administered during further operation of the drug administration device;
modifying a frequency with which a drug is administered by the drug administration device;
modifying a maximum number of drug doses possible for delivery from the drug administration device; and/or
modifying a rate with which a drug is administered by the drug administration device.

7. The method of any preceding claim, wherein measuring the at least one dispensing mechanism parameter or measuring the at least one administration parameter comprises:
measuring a speed of a motor of the drug administration device and/or a duration of operation of the motor.

8. The method of any preceding claim, wherein operating the dispensing mechanism of the drug administration device comprises:
displacing a displaceable component from a first position of the displaceable component.

9. The method of claim 8, wherein measuring the at least one dispensing mechanism parameter or the at least one administration parameter comprises:
measuring the displacement of the displaceable component.

10. The method of claim 9, wherein measuring the displacement of the displaceable component comprises using a Hall effect sensor.

11. The method of any preceding claim, wherein measuring the at least one dispensing mechanism parameter or the at least one administration parameter comprises:
measuring a flow rate of a drug administered by the drug administration device.

12. The method of any preceding claim, wherein measuring the at least one administration parameter comprises:
determining an amount of liquid present in a vicinity of an injection site.

13. The method of any preceding claim, wherein measuring the at least one administration parameter comprises:
measuring a physiological parameter, of a user of the drug administration device, associated with successful administration.

14. The method of any preceding claim, further comprising:
assessing an operational status of the drug administration device before and/or during operation of the dispensing mechanism.

15. The method of claim 14, wherein assessing the operational status of the drug administration device comprises at least one of:
analyzing a power source of the drug administration device to verify that the power source has sufficient charge for successful administration; and
sensing an angular orientation of the drug administration device relative to a user of the drug administration device and determining whether the sensed angular orientation is a proper angular orientation.

16. The method of claim 14 or claim 15, when directly or indirectly dependent on claim 8, wherein assessing the operational status of the drug administration device comprises:
moving the displaceable component of the drug administration device a predefined distance.

17. The method of any preceding claim, further comprising:
notifying a user whether the administration was successful.

18. The method of claim 17, wherein notifying the user whether the administration was successful comprises one or more of a visual feedback, an auditory feedback, and a tactile feedback.

19. The method of any preceding claim, wherein the acceptable administration parameters include a predefined range of values, and the comparing includes determining whether the measured at least one administration parameter is within the predefined range of values.

20. The method of any preceding claim, wherein the acceptable administration parameters include a predefined threshold value, and the comparing includes determining whether the measured at least one administration parameter is above the predefined threshold value.

21. The method of any preceding claim, wherein the acceptable administration parameters include a predefined threshold value, and the comparing includes determining whether the measured at least one administration parameter is below the predefined threshold value.

22. The method of any preceding claim, wherein the drug comprises at least one of infliximab, golimumab, ustekinumab, daratumumab, guselkumab, epoetin alfa, risperidone, esketamine, ketamine, and paliperidone palmitate.

23. A method for confirming administration from a drug administration device, the method comprising:
operating a dispensing mechanism of the drug administration device;
measuring at least one dispensing mechanism parameter;
determining whether the operation of the dispensing mechanism is complete based on the at least one dispensing mechanism parameter;
determining at least one physiological parameter of a user based on the at least one dispensing mechanism parameter; and
when the operation of the dispensing mechanism is determined to be complete, comparing the at least one physiological parameter with acceptable physiological parameters in order to confirm whether the administration was successful.

24. The method of claim 23, wherein measuring the at least one dispensing mechanism parameter comprises measuring a flow rate of a drug, and wherein the at least one physiological parameter is a heart rate of the user.

25. The method of claim 23 or claim 24, further comprising:
modifying further operation of the drug administration device based on the at least one dispensing mechanism parameter and/or the at least one physiological parameter.

26. The method of claim 25, further comprising:
notifying a user that the further operation of the drug administration device has been modified.

27. The method of claim 26, wherein notifying the user that the further operation of the drug administration device has been modified comprises one or more of a visual feedback, an auditory feedback, and a tactile feedback.

28. The method of any one of claims 25 to 27, wherein modifying the further operation of the drug administration device comprises:
preventing the further operation of the drug administration device when the successful administration was not confirmed.

29. The method of any one of claims 25 to 27, wherein modifying the further operation of the drug administration device comprises:
modifying a dosage volume to be administered during further operation of the drug administration device;
modifying a frequency with which a drug is administered by the drug administration device;
modifying a maximum number of drug doses possible for delivery from the drug administration device; and/or
modifying a rate with which a drug is administered by the drug administration device.

30. The method of any one of claims 23 to 29, wherein operating the dispensing mechanism of the drug administration device comprises:
displacing a displaceable component from a first position of the displaceable component.

31. The method of any one of claims 23 to 30, further comprising:
assessing an operational status of the drug administration device before operating the dispensing mechanism.

32. The method of claim 31, wherein assessing the operational status of the drug administration device comprises at least one of:
analyzing a power source of the drug administration device to verify that the power source has sufficient charge for successful administration; and
sensing an angular orientation of the drug administration device relative to a user of the drug administration device and determining whether the sensed angular orientation is a proper angular orientation.

33. The method of claim 31 or claim 32, when directly or indirectly dependent on claim 30, wherein assessing the operational status of the drug administration device comprises:
moving the displaceable component of the drug administration device a predefined distance.

34. The method of any one of claims 23 to 33, further comprising:
notifying a user whether the administration was successful.

35. The method of claim 34, wherein notifying the user whether the administration was successful comprises one or more of a visual feedback, an auditory feedback, and a tactile feedback.

36. The method of any of claims 23 to 35, wherein the drug comprises at least one of infliximab, golimumab, ustekinumab, daratumumab, guselkumab, epoetin alfa, risperidone, esketamine, ketamine, and paliperidone palmitate.

37. A drug administration system, comprising:
a drug administration device, wherein the drug administration device comprises:
a dispensing mechanism configured to dispense a drug;
at least one sensor configured to measure at least one dispensing mechanism parameter and output dispensing mechanism data relating to the at least one dispensing mechanism parameter;
wherein the system is configured to determine whether operation of the dispensing mechanism is complete based on the dispensing mechanism data; and
at least one sensor configured to measure at least one administration parameter and output administration data relating to the at least one administration parameter;
wherein the system is configured such that when the operation of the dispensing mechanism is determined to be complete, the system compares the administration data with acceptable administration data in order to confirm whether the administration was successful.

38. A drug administration device, comprising:
a dispensing mechanism configured to dispense a drug;
at least one sensor configured to measure at least one dispensing mechanism parameter and output dispensing mechanism data relating to the at least one dispensing mechanism parameter;
wherein the device is configured to determine whether operation of the dispensing mechanism is complete based on the dispensing mechanism data; and
at least one sensor configured to measure at least one administration parameter and output administration data relating to the at least one administration parameter;
wherein the device is configured such that when the operation of the dispensing mechanism is determined to be complete, the device compares the administration data with acceptable administration data in order to confirm whether the administration was successful.

39. The system of claim 37 or the device of claim 38, further comprising a first processor, wherein the first processor is configured to receive the dispensing mechanism data and to determine whether the operation of the dispensing mechanism is complete based on the dispensing mechanism data.

40. The system of claim 39 or the device of claim 39, further comprising a second processor, wherein the second processor is configured to receive the administration data and confirm whether the administration was successful when the operation of the dispensing mechanism is determined to be complete by the first processor.

41. The system of claim 40 or the device of claim 40, wherein the second processor is configured to modify further operation of the drug administration device based on the dispensing mechanism data and/or the administration data.

42. The system of claim 41 or the device of claim 41, further comprising an indicator configured to inform a user of the drug administration device that the further operation of the drug administration device has been modified.

43. The system of claim 42, or the device of claim 42 wherein the indicator is configured to provide one or more of visual feedback, auditory feedback, and tactile feedback.

44. The system of any one of claims 41 to 43 or the device of any one of claims 41 to 43, wherein the second processor being configured to modify the further operation of the drug administration device comprises:
the second processor being configured to prevent the further operation of the drug administration device when the successful administration was not confirmed.

45. The system of any one of claims 41 to 43 or the device of any one of claims 41 to 43, wherein the second processor being configured to modify the further operation of the drug administration device comprises
the second processor being configured to modify a dosage volume to be administered in any further operation of the drug administration device;
the second processor being configured to modify a frequency with which the drug is administered by the drug administration device;
the second processor being configured to modify a maximum number of drug doses possible for delivery from the drug administration device; and/or
the second processor being configured to modify a rate with which the drug is administered by the drug administration device.

46. The system of any one of claims 37 and 39 to 45 or the device of any one of claims 38 to 45, wherein the drug administration device further comprises a motor, and wherein one of the at least one dispensing sensor and the at least one administration sensor is configured to measure the speed of the motor and/or the duration of operation of the motor.

47. The system of any one of claims 37 and 39 to 46 or the device of any one of claims 38 to 46, wherein the at least one sensor configured to measure at least one dispensing mechanism parameter or the at least one sensor configured to measure at least one administration parameter comprises a Hall effect sensor.

48. The system of any one of claims 37 and 39 to 47 or the device of any one of claims 38 to 47, wherein the at least one sensor configured to measure at least one dispensing mechanism parameter or the at least one sensor configured to measure at least one administration parameter comprises a volumetric flow meter.

49. The system of any one of claims 37 and 39 to 48 or the device of any one of claims 38 to 48, wherein the at least one sensor configured to measure at least one administration parameter comprises a liquid detection sensor configured to measure the amount of liquid present in the vicinity of an injection site.

50. The system of any one of claims 37 and 39 to 49 or the device of any one of claims 38 to 49, wherein the at least one sensor configured to measure at least one administration parameter is configured to measure a physiological parameter of a user of the drug administration device, associated with successful administration.

51. The system of any one of claims 37 and 39 to 50 or the device of any one of claims 38 to 50, wherein the processor is configured to assess an operational status of the drug administration device before and/or while the drug dispensing mechanism dispenses the drug.

52. The system of claim 51 or the device of claim 51, wherein the drug administration device further comprises a power source, and wherein the processor is configured to assess the operational status of the drug administration device by verifying that the power source has sufficient charge for dispensing of the drug.

53. The system of claim 51 or 52 or the device of claim 51 or 52, wherein the dispensing mechanism further comprises a displaceable component, wherein the processor is configured to assess the operational status of the drug administration device by moving the displaceable component a predefined distance.

54. The system of any one of claims 37 and 39 to 53 or the device of any one of claims 38 to 53, further comprising an indicator configured to inform a user of the drug administration device whether the administration was successful.

55. The system of claim 54, or the device of claim 54, wherein the indicator is configured to provide visual feedback, auditory feedback, or tactile feedback.

56. The system of any of claims 37 and 39 to 55, or at device of any of claims 38 to 55, wherein the drug comprises at least one of infliximab, golimumab, ustekinumab, daratumumab, guselkumab, epoetin alfa, risperidone, esketamine, ketamine, and paliperidone palmitate.

57. A drug administration device, comprising:
a dispensing mechanism configured to dispense a drug;
at least one sensor configured to measure at least one dispensing mechanism parameter and output dispensing mechanism data relating to the at least one dispensing mechanism parameter;
wherein the device is configured to determine whether the operation of the dispensing mechanism is complete based on the dispensing mechanism data; and
a processor configured to determine at least one physiological parameter of a user of the drug administration device based on the dispensing mechanism data;
wherein the processor is configured to, when the operation of the dispensing mechanism is determined to be complete, compare the at least one physiological parameter with acceptable physiological parameters in order to confirm whether the administration was successful.

58. The device of claim 57, wherein the at least one sensor is configured to measure a flow rate of the drug, and wherein the at least one physiological parameter is a heart rate of the user.

59. The device of claim 57 or claim 58, further comprising a second processor, wherein the second processor is configured to modify further operation of the drug administration device based on the dispensing mechanism data and/or the at least one physiological parameter.

60. The device of claim 59, further comprising an indicator configured to inform a user of the drug administration device that the further operation of the drug administration device has been modified.

61. The device of claim 60, wherein the indicator is configured to provide one or more of visual feedback, auditory feedback, and tactile feedback.

62. The device of any one of claims 59 to 61, wherein the second processor being configured to modify the further operation of the drug administration device comprises:
the second processor being configured to prevent the further operation of the drug administration device when the successful administration was not confirmed.

63. The device of any one of claims 59 to 61, wherein the second processor being configured to modify the further operation of the drug administration device comprises:
the second processor being configured to modify a dosage volume to be administered in any further operation of the drug administration device;
the second processor being configured to modify a frequency with which the drug is administered by the drug administration device;
the second processor being configured to modify a maximum number of drug doses possible for delivery from the drug administration device; and/or
the second processor being configured to modify a rate at which the drug is administered by the drug administration device.

64. The device of any one of claims 57 to 63, wherein the processor is configured to assess the operational status of the drug administration device before the drug dispensing mechanism dispenses the drug.

65. The device of claim 64, wherein the drug administration device further comprises a power source, and wherein the processor is configured to assess an operational status of the drug administration device by verifying that the power source has sufficient charge for dispensing of the drug.

66. The device of claim 64 or 65, wherein the dispensing mechanism further comprises a displaceable component, wherein the processor is configured to assess the operational status of the drug administration device by moving the displaceable component a predefined distance.

67. The device of any one of claims 57 to 66, further comprising an indicator configured to inform a user of the drug administration device whether the administration was successful.

68. The device of claim 67, wherein the indicator is configured to provide visual feedback, auditory feedback, or tactile feedback.

69. The device of any of claims 57 to 67, wherein the drug comprises at least one of infliximab, golimumab, ustekinumab, daratumumab, guselkumab, epoetin alfa, risperidone, esketamine, ketamine, and paliperidone palmitate.

70. A drug administration and monitoring system, comprising:
a drug administration device configured to dispense a drug to a patient;
a monitoring device configured to log at least one delivery event of drug delivery from the drug administration device into the patient; and
a sensor configured to sense at least one patient parameter following the delivery of the drug into the patient.

71. The drug administration and monitoring system of claim 70, wherein the drug administration device, the monitoring device, and the sensor are all integrated with each other into a single device.

72. The drug administration and monitoring system of claim 70, wherein the drug administration device and the monitoring device are both integrated with each other into a single device, and the sensor is a standalone device.

73. The drug administration and monitoring system of claim 70, wherein the drug administration device, the monitoring device, and the sensor are each standalone discrete devices.

74. The drug administration and monitoring system of claim 72 or claim 73, wherein the patient sensor is configured for in vivo monitoring of the patient in real time.

75. The drug administration and monitoring system of any one of claims 70 to 74, wherein the drug administration device, the monitoring device, and the sensor are each configured to be able to be in data communication with each other.

76. The drug administration and monitoring system of any one of claims 70 to 75, wherein the monitoring device is configured to receive data pertaining to drug delivery events from the drug administration device, and to receive the at least one patient parameter from the sensor.

77. The drug administration and monitoring system of any one of claims 70 to 76, wherein the monitoring device is configured to:
determine a drug response associated with the at least one drug delivery event on the patient based on the at least one patient parameter which is sensed; and
determine and store data pertaining to a patient outcome associated with the determined drug response and the at least one drug delivery event.

78. The drug administration and monitoring system of claim 77, wherein the patient outcome is one or more of:
a time period after the at least one drug delivery event at which the drug response is sensed on the patient;
an intensity of the determined drug response at a given time or over a given time period after drug administration to the patient; and
a time duration for which the determined drug response in relation to the at least one drug delivery event.

79. The drug administration and monitoring system of any one of one of claims 70 to 79, wherein the monitoring device is further configured to:
generate a notification to the patient or a remote patient monitoring device based on the patient outcome.

80. The drug administration and monitoring system of any one of claims 70 to 79, wherein the at least one patient parameter sensed by the sensor comprises one or more of: temperature; pH level; a biomarker; glutathione level; skin thickness; subcutaneous tissue thickness; blood oxygen level; blood glucose level; blood pressure; heart rate; and metabolic rate.

81. The drug administration and monitoring system of any one of claims 70 to 80, wherein the monitoring device is further configured to check conformity of the at least one drug delivery event with a prescribed drug dosing scheme.

82. The drug administration and monitoring system of claim 81, wherein the monitoring device is further configured to generate a notification to the patient or a remote patient monitoring device if the at least one drug delivery event does not conform to the prescribed drug dosing scheme.

83. The drug administration and monitoring system of claim 83, wherein the drug dosing scheme specifies one or more of the following drug dosing parameters:
drug delivery rate;
drug delivery duration;
drug delivery volume; and
drug delivery frequency.

84. The drug administration and monitoring system of any one of claims 70 to 83, further comprising an environmental sensor configured to detect an external stimulus.

85. The drug administration and monitoring system of claim 84, wherein the environmental sensor is configured to detect one or more of: a user input to the drug administration device;
geographical location; ambient temperature; pressure; and ultraviolet radiation level.

86. The drug administration and monitoring system of claim 85, further comprising a user interface, wherein the external stimulus is a user input inputted via the user interface.

87. The drug administration and monitoring system of any one of claims 84 to 86, wherein the monitoring device is further configured to:
determine, based on the sensed at least one patient parameter and/or the external stimulus, whether a likelihood of side effects associated with the drug has increased; and
if it is determined that the likelihood of side effects has increased, generate a notification to the patient or a remote patient monitoring device if the at least one drug delivery event does not conform to the prescribed drug dosing scheme.

88. The drug administration and monitoring system of claim 87, wherein the monitoring device comprises a device indicator, wherein the drug administration device is further configured to activate the device indicator if it is determined that the likelihood of side effects has increased.

89. The drug administration and monitoring system of any one of claims 70 to 88, wherein the monitoring device is configured to provide a plurality of notifications to a patient or a remote monitoring device pertaining to the at least one drug delivery event and/or the at least one patient parameter, and wherein the plurality of notifications are provided in order according to a predefined priority order based on the at least one drug delivery event and/or the at least one patient parameter.

90. The drug administration and monitoring system of any one of the preceding claims, wherein the drug comprises at least one of infliximab, golimumab, ustekinumab, daratumumab, guselkumab, epoetin alfa, risperidone, esketamine, ketamine, and paliperidone palmitate.

91. A method of monitoring drug administration, comprising:
dispensing a drug from a drug administration device to a patient;
logging at least one drug delivery event of the drug administration device into the patient; and
sensing at least one patient parameter following delivery of drug into the patient and the logging of the at least one drug delivery event.

92. The method of claim 91, wherein the drug comprises at least one of infliximab, golimumab, ustekinumab, daratumumab, guselkumab, epoetin alfa, risperidone, esketamine, ketamine, and paliperidone palmitate.

93. A drug administration system, comprising:
a drug administration device, wherein the drug administration device comprises:
a drug holder configured to hold a drug; and
a dispensing mechanism configured to dispense the drug to a patient;
a first sensor configured to sense a patient parameter,
wherein the drug administration system is configured to locally activate the drug at a target location in the patient after the drug has been dispensed by the dispensing mechanism and administered to the patient, and
wherein the local activation is responsive to the patient parameter and an external stimulus.

94. The drug administration system of claim 93, further comprising a second sensor configured to sense the external stimulus.

95. The drug administration system of claim 94, wherein the first sensor and/or the second sensor are integral with the drug administration device.

96. The drug administration system of any one of claims 93 to 95, wherein the drug administration device is configured to delay the local activation after the drug has been administered to the patient by an amount of time such that the local activation coincides with a predicted localization time at the target location, wherein the predicted localization time is based on the sensed patient parameter and the external stimulus.

97. The drug administration system of any one of claims 93 to 96, further comprising an energy source configured to provide energy to locally activate the drug at the target location in the patient.

98. The drug administration system of claim 97, wherein an amount of energy provided by the energy source is responsive to the patient parameter and the external stimulus.

99. The drug administration system of claim 97 or 98, wherein the energy source comprises one or more of: a light source; an ultra-sound source; an electro-magnetic field source; and a radioactive material.

100. The drug administration system of any one of claims 93 to 99, wherein the drug administration device is further configured to administer a chemical activation agent to the target location in the patient to locally activate the drug.

101. The drug administration system of any one of claims 93 to 100, wherein the patient parameter sensed by the first sensor comprises one or more of: temperature; pH level; a biomarker; glutathione level; skin thickness; subcutaneous tissue thickness; blood oxygen level; blood glucose level; blood pressure; heart rate; and metabolic rate.

102. The drug administration system of any one of claims 93 to 101, wherein the external stimulus comprises one or more of: a user input; geographical location; ambient temperature; pressure; and ultraviolet radiation level.

103. The drug administration system of claim 102, further comprising a user interface, wherein the external stimulus is a user input inputted via the user interface.

104. The drug administration system of any one of claims 93 to 103, wherein the drug administration device is configured to administer the drug to the patient according to a drug dosing scheme.

105. The drug administration system of claim 104, wherein the drug dosing scheme specifies one or more of the following drug dosing parameters:
drug delivery rate;
drug delivery duration;
drug delivery volume; and
drug delivery frequency.

106. The drug administration system of claim 104 or 105, wherein the drug administration device comprises an autoinjector, and wherein the drug dosing scheme specifies one or more of the following dosing parameters:
a discharge nozzle advance depth of a discharge nozzle of the autoinjector during administration of the drug to the patient;
a discharge nozzle velocity of the discharge nozzle of the autoinjector during administration of the drug to the patient; and
a discharge nozzle acceleration of the discharge nozzle of the autoinjector during administration of the drug to the patient.

107. The drug administration system of any of claims 104 to 106, wherein the drug dosing scheme is based on the sensed patient parameter and the external stimulus.

108. The drug administration system of claim 107 when dependent on claim 106 wherein the sensed patient parameter comprises subcutaneous tissue thickness, and wherein the drug administration device is configured to adjust the discharge nozzle advance depth based on the sensed subcutaneous tissue thickness.

109. The drug administration system of any one of claims 93 to 108, wherein the drug administration system is further configured to:
determine, based on the sensed patient parameter and/or the external stimulus, whether a likelihood of side effects associated with the drug has increased; and
if it is determined that the likelihood of side effects has increased, adjust the drug dosing scheme to reduce the dosage of the drug to be administered and/or adjust an activation means of the drug administration system, the activation means being configured to locally activate the drug, to reduce local activation of the drug.

110. The drug administration system of claim 109, wherein the drug administration device further comprises a device indicator, and wherein the drug administration device is further configured to activate the device indicator if it is determined that the likelihood of side effects has increased.

111. The drug administration system of any one of claims 93 to 110, further comprising a drug capture and release mechanism configured to be implanted in a body of the patient.

112. The drug administration system of any one of claims 93 to 111, wherein the drug comprises at least one of infliximab, golimumab, ustekinumab, daratumumab, guselkumab, epoetin alfa, risperidone, esketamine, ketamine, and paliperidone palmitate.

113. A method of administering a drug to a patient using the drug administration system of any preceding claim, comprising:
dispensing the drug from the drug holder to administer the drug to the patient;
receiving data relating to the patient parameter from the first sensor and receiving data relating to the external stimulus;
comparing the received data with a lookup table; and
locally activating the drug at the target location in the patient, wherein the local activation is based on the comparison with the lookup table.

114. The method of claim 113, wherein the locally activation of the drug is delayed after the dispensing of the drug by an amount of time corresponding to a localization time determined from the lookup table.

115. The method of claim 113 or claim 114, wherein the drug comprises at least one of infliximab, golimumab, ustekinumab, daratumumab, guselkumab, epoetin alfa, risperidone, esketamine, ketamine, and paliperidone palmitate.
